(19) United States
(12) Patent Application Publication
(10) Pub. No.: US 2016/0201102 A1
(43) Pub. Date: Jul. 14, 2016

(54) Process for the Enzymatic Conversion of Lignocellulosic Biomass

(71) Applicant: NOVOZYMES A/S, Bagsvaerd (DK)

(72) Inventors: Yongming Zhu, San Jose, CA (US); Jason Holmes, Zebulon, NC (US)

(73) Assignee: Novozymes A/S, Bagsvaerd (DK)

(21) Appl. No.: 14/908,948

(22) PCT Filed: Aug. 13, 2014
(86) PCT No.: PCT/US14/50844

§ 371 Date: Jan. 29, 2016

Related U.S. Application Data
(60) Provisional application No. 61/861,139, filed on Aug. 1, 2013.

(51) Int. Cl. C12P 19/02; C12P 19/14

(52) U.S. Cl. CPC C12P 19/02; C12P 19/14; C12P 2201/00

(57) ABSTRACT

The present invention provides a process for the enzymatic conversion of pretreated lignocellulosic biomass to fermentable sugars and fermentation products, the process including the steps of saccharification of at least a portion of the cellulose and/or hemicellulose in the pretreated biomass with an enzyme mixture comprising cellulase and/or hemicellulase enzymes, to obtain a partially-hydrolyzed biomass, followed by mechanical treatment of the partially-hydrolyzed biomass, and further saccharification of the mechanically-treated, partially hydrolyzed biomass with or without further addition of an enzyme mixture.

### FIELD OF THE INVENTION

The present invention relates to the production of fermentable sugars and fermentation products from pretreated lignocellulosic feedstocks.

### BACKGROUND OF THE INVENTION

The conversion of lignocellulosic feedstocks into biofuels or other chemicals has the advantages of the ready availability of large amounts of feedstock, the desirability of avoiding burning or land filling the materials, and the cleanliness of the fuel. Wood, agricultural residues, herbaceous crops, and municipal solid wastes have been considered as feedstocks for biofuel production.

The three major constituents of lignocellulosic feedstocks, or lignocellulosic biomass, are cellulose, hemicellulose and lignin. Cellulose is a polymer of the simple sugar glucose covalently linked by beta-1,4-bonds. Hemicellulose is a branched polymer of a beta-1,4 linked backbone of xylose, xylan, plus additional sugars (arabinose, galactose, fucose, mannose) covalently linked to the xylose units. In addition to sugars, other constituents such as acetate, ferulic acid, coumaric acid, or glucuronic acid may branch from the xylan backbone via ester linkages.

The conversion of lignocellulosic feedstocks into sugars, typically, involves pretreatment followed by enzymatic hydrolysis. The pretreatment disrupts the lignocellulosic material, so enzymatic hydrolysis can take place efficiently. Under some pretreatment conditions, for example dilute acid pretreatment, most of the hemicellulose and some of the cellulose is hydrolyzed to fermentable sugars, e.g., glucose and xylose, which can be easily fermented by microbes into ethanol or catalytically converted or fermented to other chemicals.

Many microorganisms produce enzymes that hydrolyze cellulose (cellulase) and/or hemicellulose (hemicellase). Cellulase enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose, a water-soluble beta-1,4-linked dimer of glucose, from the ends of the cellulose polymer. Cellobiose is. Beta-glucosidases hydrolyze cellobiose to glucose. Hemicellulase enzymes include acetylmannan esterases, acetylxylan esterases, arabinanases, arabinofuranosidases, coumaric acid esterases, feruloyl esterases, galactosidases, glucuronidases, glucuronoyl esterases, mannanases, mannosidases, xylanases, and xylosidases.

The enzymatic hydrolysis of pretreated lignocellulosic biomass is an inefficient step in the production of fermentable sugars for biofuels and other fermentation products and its cost constitutes one of the major barriers to commercial viability. One significant problem with enzymatic hydrolysis processes is the large amount of enzyme required, which increases the cost of the process. There are several factors that contribute to the enzyme requirement, including a limitation of available or accessible surface area for the enzymes to interact with the lignocellulosic feedstock as the hydrolysis reaction progresses.

Mechanical pulping processes utilize disk refining and other methods to mechanically disrupt the fibre structure of lignocellulose. For example, U.S. Publication No. 2010/0285534 discloses combined thermochemical pretreatment and refining of lignocellulosic biomass. WO 2010/060052 A2 describes adding a refining step after a green liquor pretreatment of lignocellulose to reduce the size of the biomass to aid mixing with the green liquor. Similarly, U.S. Pat. No. 7,998,713 describes applying energy during or before an ammonium pretreatment or before or during hydrolysis of the ammonia-pretreated material in order to reduce the size of the lignocellulosic biomass.

Lee et al., 2010, Bioresource Technology 101 (19): 7218-7223, discloses an energy efficient nanofibrillation method that combines disk milling and mild hot-compressed water (HCW) treatment to improve enzymatic accessibility of Eucalyptus wood. U.S. Pat. No. 6,267,841 discloses a low energy mechanical pulping process which employs an enzyme treatment stage between two low energy refining stages (conducted at less than 10 or 20 hpd/tonne). U.S. Publication No. 2012/0135506 describes an energy-efficient process for producing microfibrillated cellulose at high consistency in which cellulose fibres are subject to an enzymatic treatment, a first mechanical treatment, a second enzymatic treatment, and a second mechanical treatment.

It would be advantageous to the art to be able to improve the efficiency of enzymatic hydrolysis of lignocellulosic biomass. The present invention relates to processes for mechanically assisted hydrolysis of lignocellulosic biomass for the production of fermentable sugars.

### SUMMARY OF THE INVENTION

In a first aspect, the invention provides a process for producing fermentable sugars from biomass, comprising
a. preparation of a biomass-enzyme mixture of (i) pretreated lignocellulosic biomass containing cellulose and/or hemicellulose and (ii) an enzyme composition comprising cellulase and/or hemicellulase enzymes;
b. a first saccharification of the biomass-enzyme mixture for a sufficient time to achieve hydrolysis of at least about 10% of the cellulose and/or hemicellulose and produce partially-hydrolyzed biomass and hydrolysate liquor;
c. mechanical treatment of the partially-hydrolyzed biomass to produce mechanically-disrupted, partially-hydrolyzed biomass; and
d. a second saccharification of the mechanically-disrupted, partially-hydrolyzed biomass for a sufficient time to achieve hydrolysis of at about 60% to about 100% of the cellulose and/or hemicellulose present in the pretreated lignocellulosic biomass to fermentable sugars.

In some embodiments, the second saccharification of the mechanically-disrupted, partially-hydrolyzed biomass is conducted without an additional dose of cellulase and/or hemicellulose enzymes. In other embodiments, the second saccharification of the mechanically-disrupted, partially-hydrolyzed biomass is conducted with an additional dose of cellulase and/or hemicellulase enzymes.

In some embodiments, the mechanical treatment is selected from the group consisting of refining, milling, crushing, grinding, shredding, extrusion, beating, or combinations thereof. For example, the mechanical treatment may be refining conducted so as to provide a refining energy of from about 50 to about 500 kWh per dry tonne of biomass.

In other embodiments, one or more additional mechanical treatment is applied to the mechanically-disrupted, partially-hydrolyzed biomass after the second saccharification, each additional mechanical treatment being followed by an additional saccharification, which may be conducted with or without an additional dose of cellulase and/or hemicellulose enzymes.

In some embodiments, a solids liquid separation step is conducted after the first saccharification step and before the mechanical treatment step. In other embodiments, the mechanically-treated, partially-hydrolyzed biomass is recombined with the hydrolysate liquor collected from the solids-liquid separation prior to the second saccharification.

In still other embodiments, the pretreated lignocellulosic biomass is produced by one or more pretreatment method, including steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation. In preferred embodiments, the pretreatment method is hot water pretreatment, steam pretreatment, dilute acid pretreatment, wet oxidation, wet explosion pretreatment with organic solvents, biological pretreatment, supercritical CO₂ pretreatment, or ozone pretreatment.

The cellulase enzyme used in the process of the present invention may be a cellobiohydrolase, an endoglucanase, a beta-glucosidase, or mixtures thereof. The cellulase enzyme may further comprise one or more (e.g., several) proteins selected from the group consisting of an AA9 polypeptide having cellulolytic enhancing activity, an expansin, a ligninolytic enzyme, an oxidoreductase, a pectinase, a protease, and a swollenin.

The hemicellulose enzyme used in the process of the present invention may be an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase, or any combination thereof.

In a second aspect, the invention provides a process for producing a fermentation product, comprising the processes for producing fermentable sugars described above, fermenting the fermentable sugars with one or more fermenting microorganisms to produce the fermentation product; and recovering the fermentation product from the fermentation.

In some embodiments, the step of fermenting the fermentable sugars is conducted simultaneously with either or both the first or second saccharifications in a simultaneous saccharification and fermentation.

In some embodiments, the fermentation product is an alcohol, an alkane, a cycloalkane, an alkene, an amino acid, a gas, isoprene, a ketone, an organic acid, or polyketide. For example, the alcohol may be ethanol, n-butanol, isobutanol, methanol, arabinitol, butanediol, ethylene glycol, glycerin, glycerol, 1,3-propanediol, sorbitol, or xylitol; the alkane may be pentane, hexane, heptane, octane, nonane, decane, undecane, or dodecane; the cycloalkane may be cyclopentane, cyclohexane, cycloheptane, or cyclooctane; the alkene may be pentene, hexene, heptene, or octane; the amino acid may be aspartic acid, glutamic acid, glycine, lysine, serine, or threonine; the gas is methane, hydrogen gas, carbon dioxide, or carbon monoxide; the ketone may be acetone; and the organic acid may be acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. In a preferred embodiment, the fermentation product is an alcohol, which may be ethanol, n-butanol, or isobutanol.

In a third aspect, the invention provides a process for producing a fermentation product comprising
a. pretreatment of lignocellulosic biomass containing cellulose and/or hemicellulose by hot water pretreatment, steam pretreatment, dilute acid pretreatment, wet oxidation, wet explosion pretreatment with organic solvents, biological pretreatment, supercritical CO₂ pretreatment, or ozone pretreatment
b. preparation of a biomass-enzyme mixture comprising (i) the pretreated lignocellulosic biomass of step (a) and (ii) an enzyme composition comprising cellulase enzymes and/or hemicellulase enzymes;
c. a first saccharification of the biomass-enzyme mixture from step (b) for a sufficient time to achieve hydrolysis of at least about 10% of the cellulose and/or hemicellulose and produce partially-hydrolyzed biomass and hydrolysate liquor;
d. mechanical treatment of the partially-hydrolyzed biomass produced by step (c) to produce mechanically-disrupted, partially-hydrolyzed biomass;
e. a second saccharification of the mechanically-disrupted, partially-hydrolyzed biomass produced by step (d) for a sufficient time to achieve hydrolysis of at least about 60% of the cellulose and/or hemicellulose present in the pretreated lignocellulosic biomass to fermentable sugars;
f. fermenting the fermentable sugars produced in step (e) with one or more fermenting microorganisms to produce the fermentation product; and
g. recovering the fermentation product from the fermentation.

### BRIEF DESCRIPTION OF DRAWINGS

FIG. 1 shows the effect of mechanical treatment of partially-hydrolyzed biomass on the subsequent saccharification of cellulose in the biomass. Pretreated biomass was incubated with an enzyme composition (2 mg protein/gm biomass) comprising cellulases and hemicellulases for 3 days, the partially-hydrolyzed biomass was separated from the hydrolysate liquor and refined with a PFI laboratory refiner for 5000, 10000, 15000 or 20000 counts, recombined with the hydrolysate liquor and subjected to a second saccharification for 2 days. Fractional glucan conversion (cellulose plus beta-glucan) was determined by HPLC analysis of the released sugars.

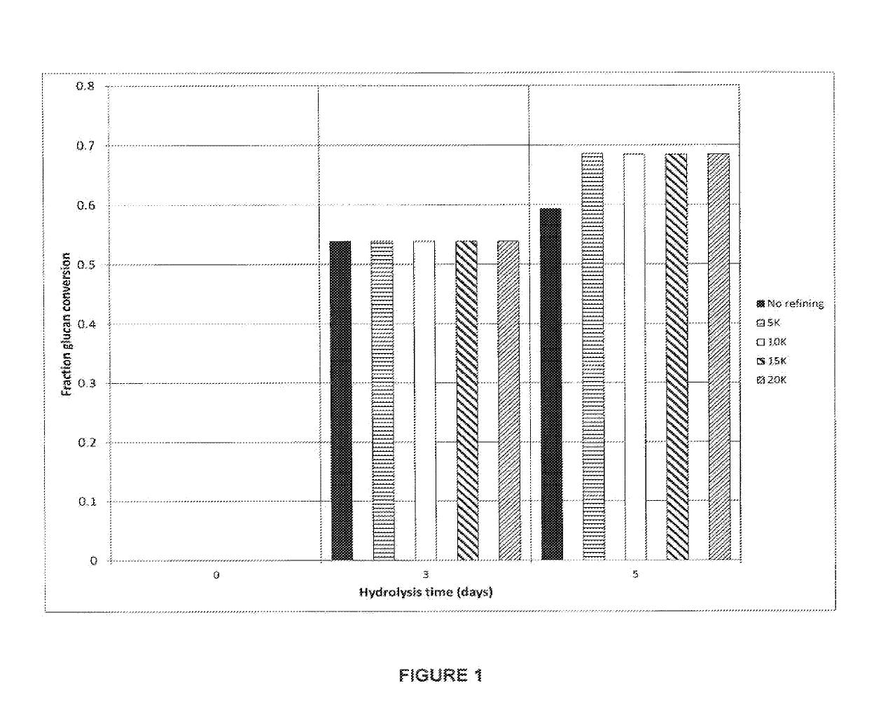

FIG. 2 shows the effect of mechanical treatment of partially-hydrolyzed biomass on the subsequent saccharification of xylose in the biomass. Pretreated biomass was incubated with an enzyme composition (2 mg protein/gm biomass) comprising cellulases and hemicellulases for 3 days, the partially-hydrolyzed biomass was separated from the hydrolysate liquor and refined with a PFI laboratory refiner for 5000, 10000, 15000 or 20000 counts, recombined with the hydrolysate liquor and subjected to a second saccharification for 2 days. Fractional xylan conversion was determined by measuring the released sugars by HPLC.

FIG. 3 shows the effect of mechanical treatment of partially-hydrolyzed biomass on the subsequent saccharification of cellulose and xylan in the biomass. Pretreated biomass was incubated with an enzyme composition (2 mg protein/gm biomass) comprising cellulases and hemicellulases for 3 days, the partially-hydrolyzed biomass was separated from the hydrolysate liquor and refined with a PFI laboratory refiner for 5000, 10000, 15000 or 20000 counts, recombined with the hydrolysate liquor and subjected to a second saccharification for 2 days. Fractional glucan (cellulose+beta-glucan) and xylan conversion was determined by HPLC analysis of the released sugars.

FIG. 4 shows the effect of mechanical treatment of partially-hydrolyzed biomass on the subsequent saccharification with and without an additional dose of the enzyme composition after mechanical treatment. Pretreated biomass was incubated with an enzyme composition comprising cellulases and hemicellulases (2 mg protein/gm biomass) for 3 days, the partially-hydrolyzed biomass was separated from the hydrolysate liquor and refined with a PFI laboratory refiner for 5000, 10000, 15000, or 20000 counts, recombined with the hydrolysate liquor and subjected to a second saccharification for 2 days with or without additional enzyme composition (1 mg protein/gm biomass). Fractional glucan (cellulose+beta-glucan) was determined by HPLC analysis of the released sugars.

FIG. 5 shows the effect of a second mechanical treatment of partially-hydrolyzed biomass on the subsequent saccharification. Pretreated biomass was incubated with an enzyme composition comprising cellulases and hemicellulases (2 mg protein/gm biomass) for 3 days, the partially-hydrolyzed biomass was separated from the hydrolysate liquor and refined with a PFI laboratory refiner for 5000 counts, recombined with the hydrolysate liquor and subjected to a second saccharification for 2 days. The mechanically-treated, partially-hydrolyzed biomass was either recombined with the hydrolysate liquor and subjected to a third saccharification for 2 days or subjected to a second mechanical treatment with a PFI laboratory refiner for 5000, 10000, or 20000 counts, recombined with the hydrolysate liquor and subjected to a third saccharification for 2 days. Fractional glucan (cellulose+beta-glucan) was determined by HPLC analysis of the released sugars.

FIG. 6 shows the effect of a second mechanical treatment of partially-hydrolyzed biomass on the subsequent saccharification. Pretreated biomass was incubated with an enzyme composition comprising cellulases and hemicellulases (2 mg protein/gm biomass) for 3 days, the partially-hydrolyzed biomass was separated from the hydrolysate liquor and refined with a PFI laboratory refiner for 5000 counts, recombined with the hydrolysate liquor and subjected to a second saccharification for 2 days. The mechanically-treated, partially-hydrolyzed biomass was either recombined with the hydrolysate liquor and subjected to a third saccharification for 2 days or subjected to a second mechanical treatment with a PFI laboratory refiner for 20000 counts, recombined with the hydrolysate liquor and subjected to a third saccharification for 2 days. Fractional glucan (cellulose+beta-glucan) was determined by HPLC analysis of the released sugars.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. Acetylxylan esterase activity may be determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C. An acetyl xylan esterase may be a member of Carbohydrate Esterase Family 1, 2, 3, 4, 5, 6, 7, 12 or 15. Examples of acetylxylan esterases useful in the processes of the present invention include, but are not limited to, acetylxylan esterases from Aspergillus aculeatus (WO 2010/108918), Chaetomium globosum (UniProt:Q2GWX4), Chaetomium gracile (GeneSeqP:AAB82124), Humicola insolens DSM 1800 (WO 2009/073709), Hypocrea jecorina (WO 2005/001036), Myceliophtera thermophila (WO 2010/014880), Neurospora crassa (UniProt:q7s259), Phaeosphaeria nodorum (UniProt:Q0UHJ1), and Thielavia terrestris NRRL 8126 (WO 2009/042846).

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. Alpha-L-arabinofuranosidase activity may be determined using 5 mg of medium viscosity wheat arabinoxylan (Megazyme International Ireland, Ltd., Bray, Co. Wicklow, Ireland) per mL at pH 5, 40° C. for 30 minutes followed by arabinose analysis by AMINEX® HPX-87H column chromatography (Bio-Rad Laboratories, Inc., Hercules, Calif., USA). An alpha-L-arabinofuranosidase may comprise a catalytic domain of GH Family 3, 10, 43, 51, 54, or 62. Examples of arabinofuranosidases useful in the processes of the present invention include, but are not limited to, arabinofuranosidases from Aspergillus niger (GeneSeqP:AAR94170), Humicola insolens DSM 1800 (WO 2006/114094 and WO 2009/073383), and M. giganteus (WO 2006/114094).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. Alpha-glucuronidase activity may be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C. An alpha-glucuronidase may comprise a catalytic domain of GH Family 4 or 67. Examples of alpha-glucuronidases useful in the processes of the present invention include, but are not limited to, alpha-glucuronidases from Aspergillus clavatus (UniProt:alcc12), Aspergillus fumigatus (SwissProt:Q4WW45), Aspergillus niger (UniProt:Q96WX9), Aspergillus terreus (SwissProt:Q0CJP9), Humicola insolens (WO 2010/014706), Penicillium aurantiogriseum (WO 2009/068565), Talaromyces emersonii (UniProt:Q8X211), and Trichoderma reesei (UniProt:Q99024).

Auxilliary Activity 9: The term “Auxilliary Activity 9” or “AA9” means a polypeptide classified as lytic polysaccharide monooxygenases (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into Glycoside Hydrolase Family 61 according to Henrissat, 1991, Biochem. J. 280: 309-316, and Henrissat and Bairoch, 1996, Biochem. J. 316: 695-696. The enzymes in this family were originally classified as a glycoside hydrolase family based on measurement of very weak endo-1,4-beta-D-glucanase activity in one family member.

Examples of AA9 polypeptides useful in the processes of the present invention include, but are not limited to, AA9 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290), Myceliophthora thermophila (WO 2009/085935, WO 2009/085859, WO 2009/085864, and WO 2009/085868), Aspergillus fumigatus (WO 2010/138754), Penicillium pinophilum (WO 2011/005867), Thermoascus sp. (WO 2011/039319), Penicillium sp. (WO 2011/041397), Thermoascus crustaceous (WO 2011/041504), Aspergillus aculeatus (WO 2012/125925), Thermomyces lanuginosus (WO 2012/113340, WO 12/129699, and WO 2012/130964), Aurantiporus alborubescens (WO 2012/122477), Trichophaea saccata (WO 2012/122477), Penicillium thomii (WO 2012/122477), Talaromyces stipitatus (WO 2012/135659), Humicola insolens (WO 2012/146171), Malbranchea cinnamomea (WO 2012/101206), Talaromyces leycettanus (WO 2012/101206), and Chaetomium thermophilum (WO 2012/101206).

In one aspect, the AA9 polypeptide is used in the presence of a soluble activating divalent metal cation according to WO 2008/151043, e.g., manganese or copper.

In another aspect, the AA9 polypeptide is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (WO 2012/021394, WO 2012/021395, WO 2012/021396, WO 2012/021399, WO 2012/021400, WO 2012/021401, WO 2012/021408, and WO 2012/021410). The term “liquor” means the solution phase, either aqueous, organic, or a combination thereof, arising from pre-treatment and/or hydrolysis of a lignocellulose and/or hemicellulose material in a slurry, or monosaccharides thereof, e.g., xylose, arabinose, mannose, etc., under conditions as described herein, and the soluble contents thereof. A liquor for cellulolytic enhancement by an AA9 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and an AA9 polypeptide during hydrolysis of a cellulosic substrate by a cellulase enzyme. The liquor can be separated from the treated material using a method standard in the art, such as filtration, sedimentation, or centrifugation.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. Beta-glucosidase activity is preferably determined using p-nitrophenyl-beta-D-glucopyranoside as substrate according to the procedure of Venturi et al., 2002, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate. A beta-glucosidase may comprise a catalytic domain of GH Family 1, 3, 5, 9, 30 or 116. Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 2002/095014), Penicillium brasilianum IBT 20888 (WO 2007/019442 and WO 2010/088387), Thielavia terrestris (WO 2011/035029), and Trichophaea saccata (WO 2007/019442). The Aspergillus oryzae beta-glucosidase can be obtained according to WO 2002/095014. The Aspergillus fumigatus beta-glucosidase can be obtained according to WO 2005/047499. The Penicillium brasilianum beta-glucosidase can be obtained according to WO 2007/019442. The Aspergillus niger beta-glucosidase can be obtained according to Dan et al., 2000, J. Biol. Chem. 275: 4973-4980. The Aspergillus aculeatus beta-glucosidase can be obtained according to Kawaguchi et al., 1996, Gene 173: 287-288.

Examples of other beta-glucosidases useful in the present invention include a chimeric beta-glucosidase produced from Aspergillus fumigatus and Aspergillus aculeatus beta-glucosidases (WO 2013/089889).

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. Beta-xylosidase activity is preferably determined using 1 mM p-nitrophenyl-beta-D-xyloside as substrate. One unit of beta-xylosidase is defined as 1.0 μmole of p-nitrophenolate anion produced per minute at 40° C., pH 5 from 1 mM p-nitrophenyl-beta-D-xyloside. A beta-xylosidase may comprise a catalytic domain of GH Family 1, 3, 30, 39, 43, 51, 52, 116 or 120. Examples of beta-xylosidases useful in the processes of the present invention include, but are not limited to, beta-xylosidases from Neurospora crassa (SwissProt:Q7SOW4), Trichoderma reesei (UniProtKB/TrEMBL:Q92458), Talaromyces emersonii (SwissProt:Q8X212), and Talaromyces thermophilus GH11 (WO 2012/13095).

Biomass: The term “biomass” means any herbaceous, plant or plant-derived material comprising cellulose. Biomass includes, for example, the stems, leaves, hulls, husks, and cobs of plants, as well as the leaves, branches, and wood of trees. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose.

Biomass can be, but is not limited to, agricultural residue (including sugar cane bagasse, corn stover, wheat straw, barley straw, rice straw, oat straw, canola straw, and soybean stover), herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York).

Carbohydrate binding module: The term “carbohydrate binding module” means the non-catalytic region within a carbohydrate-active enzyme that provides carbohydrate-binding activity (Boraston et al., 2004, Biochem. J. 383: 769-781). A majority of known carbohydrate binding modules (CBMs) are contiguous amino acid sequences with a discrete fold. The carbohydrate binding module (CBM) is typically found either at the N-terminal or at the C-terminal extremity of an enzyme. CBMs are typically found either at the N-terminal or at the C-terminal extremity of a variety of enzymes involved in the degradation of carbohydrate substrates, including cellulases, hemicellulases, glucanases, amylases, glucoamylases, chitinases and the like. CBMs can recognize and bind to crystalline cellulose, non-crystalline cellulose, chitin, beta-1,3 glucans, mixed beta-1,3-1,4 glucans, xylan, mannan, galactan, and starch. CBMs assume a variety of structures that govern their substrate binding affinities and can therefore also be classified into Families based on their structural and functional relationships. To date there are 67 known CBM Families (see URL cazy.org/fam/acc_CDM.html).

Catalytic domain: The term “catalytic domain” means the region of an enzyme containing the catalytic machinery of the enzyme. The catalytic domain of cellulase enzymes, hemicellulase enzymes, and related enzymes and proteins are defined both by the Joint Commission on Biochemical Nomenclature of the International Union of Biochemistry and Molecular Biology (Published in Enzyme Nomenclature 1992, Academic Press, San Diego, Calif., ISBN 0-12-227164-5; with supplements in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650, each of which are incorporated herein by reference; also see: chem.qmul.ac.uk/iubmb/enzyme/) and also by the Glycoside Hydrolase (GH) Families as defined by the CAZy system which is accepted as a standard nomenclature for Glycoside Hydrolase (GH) enzymes (Coutinho, P. M. & Henrissat, B., 1999, “Carbohydrate-active enzymes: an integrated database approach.” In Recent Advances in Carbohydrate Bioengineering, H. J. Gilbert, G. Davies, B. Henrissat and B. Svensson eds., The Royal Society of Chemistry, Cambridge, pp. 3-12, which is incorporated herein by reference; also see www.cazy.org/Glycoside-Hydrolases.html) and is familiar to those skilled in the art.

In addition to the above nomenclature systems, polysaccharide-degrading enzymes have been, and continue to be, identified by an earlier nomenclature system whereby each successive carbohydrate active enzyme identified or isolated from a given source organism is numbered sequentially in the order of discovery. For example, the cellulose-degrading enzyme system produced by the fungus Trichoderma reesei include a GH7 cellobiohydrolase (Cel7A or CBH1), a GH6 cellobiohydrolase (Cel6A or CBH2), a GH7 endoglucanase (Cel7B or EG1), a GH5 endoglucanase (Cel5A or EG2), two GH11 xylanases (Xyn1 or Xyl11A, Xyn2 or Xyl11B),

Cellobiohydrolase: The term “cellobiohydrolase” or “CBH” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity may be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581. Cellobiohydrolases may comprise a catalytic domain of GH Family 5, 6, 7, 9 or 48. Examples of cellobiohydrolases useful in the present invention include, but are not limited to, Aspergillus aculeatus cellobiohydrolase II (WO 2011/059740), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (GenBank:AY690482), Talaromyces emersonii cellobiohydrolase I (GenBank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic enzyme activity include: (1) measuring the total cellulolytic activity, and (2) measuring the individual cellulolytic activities (endoglucanases, cellobiohydrolases, and beta-glucosidases) as reviewed in Zhang et al., 2006, Biotechnology Advances 24: 452-481. Total cellulolytic activity may be measured using insoluble substrates, including Whatman No 1 filter paper, microcrystalline cellulose, bacterial cellulose, algal cellulose, cotton, and pretreated lignocellulose. The most common total cellulolytic activity assay is the filter paper assay using Whatman No 1 filter paper as the substrate. The assay was established by the International Union of Pure and Applied Chemistry (IUPAC) (Ghose, 1987, Pure Appl. Chem. 59: 257-68).

For purposes of the present invention, cellulolytic enzyme activity may be determined by measuring the increase in the production/release of sugars during the enzymatic hydrolysis of a cellulosic material by cellulolytic enzyme(s) under the following conditions: 1-50 mg of cellulolytic enzyme protein/g of cellulose for 3-7 days at a suitable temperature, e.g., such as 40° C.-80° C., e.g., 40° C., 50° C., 55° C., 60° C., 65° C., 70° C., or 80° C., and a suitable pH, such as 4-9, e.g., 4.0, 4.5, 5.0, 5.5, 6.0, 6.5, 7.0, 7.5, 8.0, 8.5, or 9.0 compared to a control hydrolysis without addition of cellulolytic enzyme protein. Typical conditions are 1 ml reactions, washed or unwashed lignocellulosic biomass (5% w/v insoluble solids), 72 hours, sugar analysis by AMINEX® HPX-87H column (Bio-Rad Laboratories, Inc., Hercules, Calif., USA).

Cellulosic material: The term “cellulosic material” means any material containing cellulose. Cellulose is a homopolymer of anhydrocellobiose and thus a linear beta-(1-4)-D-glucan. Although generally polymorphous, cellulose is found in plant tissue primarily as an insoluble crystalline matrix of parallel glucan chains. Cellulose is generally found, for example, in the stems, leaves, hulls, husks, and cobs of plants or leaves, branches, and wood of trees. The cellulosic material can be, but is not limited to, biomass, municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of biomass or lignocellulose—i.e, plant cell wall material containing lignin, cellulose, and hemicellulose in a mixed matrix. The cellulosic material is any biomass material including, but not limited to, lignocellulose, (comprising cellulose, hemicellulose, and lignin) and pretreated lignocellulose.

In one aspect, the cellulosic material is agricultural residue, herbaceous material (including energy crops), municipal solid waste, pulp and paper mill residue, waste paper, or wood (including forestry residue).

In another aspect, the cellulosic material is arundo, bagasse, bamboo, corn cob, corn fiber, corn stover, miscanthus, rice straw, switchgrass, or wheat straw.

In another aspect, the cellulosic material is aspen, eucalyptus, fir, pine, poplar, spruce, or willow.

In another aspect, the cellulosic material is algal cellulose, bacterial cellulose, cotton linter, filter paper, microcrystalline cellulose (e.g., AVICEL®), or phosphoric-acid treated cellulose.

In another aspect, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein.

Endoglucanase: The term “endoglucanase” or “EG” means an endo-1,4-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity may be determined using carboxymethyl cellulose (CMC) as substrate according to the procedure of Ghose, 1987, Pure and Appl. Chem. 59: 257-268. An endoglucanase may comprise a catalytic domain of GH Family 5, 6, 7, 8, 9, 10, 12, 16, 44, 45, 48, 51, 74 and 124.

Examples of bacterial endoglucanases that can be used in the processes of the present invention, include, but are not limited to, Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases that can be used in the present invention, include, but are not limited to, Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, GenBank:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (GenBank:XM_324477), Humicola insolens endoglucanase V, Myceliophthora thermophila CBS 117.65 endoglucanase, Thermoascus aurantiacus endoglucanase I (GenBank:AF487830) and Trichoderma reesei strain No. VTT-D-80133 endoglucanase (GenBank:M15665).

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II. Feruloyl esterase activity may be determined using 0.5 mM p-nitrophenylferulate as substrate. One unit of feruloyl esterase equals the amount of enzyme capable of releasing 1 μmole of p-nitrophenolate anion per minute at pH 5, 25° C. Examples of feruloyl esterases (ferulic acid esterases) useful in the processes of the present invention include, but are not limited to, feruloyl esterases form Humicola insolens DSM 1800 (WO 2009/076122), Neosartorya fischeri (UniProt:A1D9T4), Neurospora crassa (UniProt:Q9HGR3), Penicillium aurantiogriseum (WO 2009/127729), and Thielavia terrestris (WO 2010/053838 and WO 2010/065448).

Hemicellulose: The term “hemicellulose” or “hemicellulosic material” refers to one or more members of a heterogeneous group of branched and linear polysaccharides that are bound via hydrogen bonds to the cellulose microfibrils in the plant cell wall, crosslinking them into a robust network. Hemicelluloses are also covalently attached to lignin via ester bonds forming, together with cellulose, a highly complex structure.

Hemicellulolytic enzyme or hemicellulase: The term “hemicellulolytic enzyme” or “hemicellulase” means one or more (e.g., several) enzymes that hydrolyze hemicellulose or hemicellulosic material. See, for example, Shallom and Shoham, Current Opinion In Microbiology, 2003, 6(3): 219-228). The variable structure and organization of hemicelluloses requires the concerted action of many enzymes for its complete degradation. Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., such as 40° C.-80° C., e.g., 40° C., 50° C., 55° C., 60° C., 65° C., 70° C., or 80° C., and a suitable pH, such as 4-9, e.g., 4.0, 4.5, 5.0, 5.5, 6.0, 6.5, 7.0, 7.5, 8.0, 8.5, or 9.0.

Mechanical Disruption: The term “mechanical disruption” means a process by which mechanical energy is applied to a cellulosic material (including biomass, lignocellulose or pretreated lignocellulose) in order to disrupt fibre structure and/or increase surface area. Cellulosic material thus treated is “mechanically disrupted”.

Polypeptide having cellulolytic enhancing activity: The term “polypeptide having cellulolytic enhancing activity” means an AA9 polypeptide that catalyzes the enhancement of the hydrolysis of a cellulosic material by an enzyme having cellulolytic activity. Cellulolytic enhancing activity is preferably determined by measuring the increase in reducing sugars or the increase of the total of cellobiose and glucose from the hydrolysis of a cellulosic material by cellulolytic enzyme(s) under the following conditions: 1-50 mg of total protein/g of cellulose in pretreated lignocellullose, wherein total protein is comprised of 50-99.5% w/w cellulolytic enzyme protein and 0.5-50% w/w protein of an AA9 polypeptide having cellulolytic enhancing activity for 1-7 days at a suitable temperature, e.g., such as 40° C.-80° C., e.g., 40° C., 50° C., 55° C., 60° C., 65° C., 70° C., or 80° C., and a suitable pH, such as 4-9, e.g., 4.0, 4.5, 5.0, 5.5, 6.0, 6.5, 7.0, 7.5, 8.0, 8.5, or 9.0, compared to a control hydrolysis with equal total protein loading without cellulolytic enhancing activity (1-50 mg of cellulolytic protein/g of cellulose in pretreated lignocellulose).

In one aspect, the cellulolytic enhancing activity of an AA9 polypeptide is determined using a mixture of CELLUCLAST® 1.5 L (Novozymes A/S, Bagsværd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 02/095014) of cellulase protein loading is used as the source of the cellulolytic activity.

In another aspect, AA9 polypeptide enhancing activity is determined according to WO 2013/028928 for high temperature compositions.

The AA9 polypeptides having cellulolytic enhancing activity enhance the hydrolysis of a cellulosic material catalyzed by an enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Pretreated Lignocellulose: The term “pretreated lignocellulose” means a cellulosic material derived from biomass or lignocellulose by hot water pretreatment, steam pretreatment, dilute acid pretreatment, wet oxidation, wet explosion pretreatment with organic solvents, biological pretreatment, supercritical CO₂ pretreatment, ozone pretreatment or any pretreatment otherwise described herein or known in the art.

Refining: The term “refining” means the treatment of cellulosic material (including biomass, lignocellulose and pretreated lignocellulose), in the presence of water with metallic bars or plates. The plates or bars are grooved to facilitate fiber transportation through the refining machine. Refining may result in one or more of the following changes in fibre structure: cutting and shortening, fibrillation, swelling, redistribution of hemicelluloses from the interior of the fiber to the exterior, and abrasion of the fibre surface at the molecular level.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the processes of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270.

For purposes of the present invention, xylan degrading activity may be determined by measuring the production/release of xylose during the enzymatic hydrolysis of a xylan-containing material including, but not limited to, biomass, lignocellulose, and pretreated lignocellulose, under the following conditions: 0.1-50 mg of xylan-degrading enzyme protein/g of pretreated lignocellulose for 3-7 days at a suitable temperature, e.g., such as 40° C.-80° C., e.g., 40° C., 50° C., 55° C., 60° C., 65° C., 70° C., or 80° C., and a suitable pH, such as 4-9, e.g., 4.0, 4.5, 5.0, 5.5, 6.0, 6.5, 7.0, 7.5, 8.0, 8.5, or 9.0 compared to a control hydrolysis without addition of xylan degrading enzyme. Typical conditions are 1 ml reactions, washed or unwashed xylan-containing material (5% w/v insoluble solids), 72 hours, sugar analysis by AMINEX® HPX-87H column (Bio-Rad Laboratories, Inc., Hercules, Calif., USA).

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. Xylanase activity may be determined by measuring the increase in hydrolysis of birchwood xylan or wheat arabinoxylan (Sigma Chemical Co., Inc., St. Louis, Mo., USA) by xylanase enzyme(s) under the following typical conditions: 5 mg/ml substrate (total solids), 5 mg of xylanase protein/g of substrate, 50 mM sodium acetate pH 5, 50° C., 24 hours, sugar analysis using p-hydroxybenzoic acid hydrazide (PHBAH) assay as described by Lever, 1972, Anal. Biochem. 47: 273-279. Xylanase activity may also determined with 0.2% AZCL-arabinoxylan (Sigma Chemical Co., Inc., St. Louis, Mo., USA). One unit of xylanase activity is defined as 1.0 μmole of azurine produced per minute at 37° C., pH 6. Examples of xylanases useful in the processes of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Talaromyces lanuginosus GH11 (WO 2012/130965), Talaromyces thermophilus GH11 (WO 2012/13095), Thielavia terrestris NRRL 8126 (WO 2009/079210), and Trichophaea saccata GH10 (WO 2011/057083).

### DETAILED DESCRIPTION OF THE INVENTION

The following description is of embodiments by way of example only and without limitation to the combination of features necessary for carrying the invention into effect. The headings provided are not meant to be limiting of the various embodiments of the invention. Terms such as “comprises,” “comprising,” “comprise,” “includes,” “including,” and “include” are not meant to be limiting. In addition, the use of the singular includes the plural, and “or” means “and/or” unless otherwise stated. Unless otherwise defined herein, all technical and scientific terms used herein have the same meaning as commonly understood by one of ordinary skill in the art.

In a first aspect, the invention provides a process for producing fermentable sugars from biomass, comprising:
a. preparation of a biomass-enzyme mixture of (i) pretreated lignocellulosic biomass containing cellulose and/or hemicellulose and (ii) an enzyme composition comprising cellulase and/or hemicellulase enzymes;
b. a first saccharification of the biomass-enzyme mixture for a sufficient time to achieve hydrolysis of at least about 10% of the cellulose and/or hemicellulose and to produce partially-hydrolyzed biomass and hydrolysate liquor;
c. mechanical treatment of the partially-hydrolyzed biomass to produce mechanically-disrupted, partially-hydrolyzed biomass; and
d. a second saccharification of the mechanically-disrupted, partially-hydrolyzed biomass for a sufficient time to achieve hydrolysis of at least about 60% to about 100% of the cellulose and/or hemicellulose present in the pretreated lignocellulosic biomass to fermentable sugars.

In some embodiments, the second saccharification of the mechanically-disrupted, partially-hydrolyzed biomass is conducted without an additional dose of cellulase and/or hemicellulose enzymes. In other embodiments, the second saccharification of the mechanically-disrupted, partially-hydrolyzed biomass is conducted with an additional dose of cellulase and/or hemicellulase enzymes.

In some embodiments, the mechanical treatment is selected from the group consisting of refining, milling, crushing, grinding, shredding, extrusion, beating, or combinations thereof. In some embodiments, the mechanical treatment is refining conducted so as to provide a refining energy of from about 50 to about 500 kWh per dry tonne of biomass, or any range therebetween. In other embodiments, one or more additional mechanical treatments is applied to the mechanically-disrupted, partially-hydrolyzed biomass after the second saccharification, each additional mechanical treatment being followed by an additional saccharification, which may be conducted with or without an additional dose of cellulase and/or hemicellulose enzymes.

In some embodiments, a solids liquid separation step is conducted after the first saccharification step and before the mechanical treatment step. In other embodiments, the mechanically-treated, partially-hydrolyzed biomass is recombined with the hydrolysate liquor collected from the solids-liquid separation prior to the second saccharification.

In still other embodiments, the pretreated lignocellulosic biomass is produced by one or more pretreatment methods, including steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation. In preferred embodiments, the pretreatment method is hot water pretreatment, steam pretreatment, dilute acid pretreatment, wet oxidation, wet explosion pretreatment with organic solvents, biological pretreatment, supercritical CO₂ pretreatment, or ozone pretreatment.

The cellulase enzyme used in the process of the present invention may be a cellobiohydrolase, an endoglucanase, a beta-glucosidase, or mixtures thereof. The cellulase enzyme may further comprise one or more (e.g., several) proteins selected from the group consisting of an AA9 polypeptide having cellulolytic enhancing activity, an expansin, a ligninolytic enzyme, an oxidoreductase, a pectinase, a protease, and a swollenin.

The hemicellulose enzyme used in the process of the present invention may be an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase, or any combination thereof.

In a second aspect, the invention provides a process for producing a fermentation product, comprising the processes for producing fermentable sugars described above, fermenting the fermentable sugars with one or more fermenting microorganisms to produce the fermentation product; and recovering the fermentation product from the fermentation.

In some embodiments, the step of fermenting the fermentable sugars is conducted simultaneously with either or both the first or second saccharifications in a simultaneous saccharification and fermentation.

In some embodiments, the fermentation product is an alcohol, an alkane, a cycloalkane, an alkene, an amino acid, a gas, isoprene, a ketone, an organic acid, or a polyketide. For example, the alcohol may be ethanol, n-butanol, isobutanol, methanol, arabinitol, butanediol, ethylene glycol, glycerin, glycerol, 1,3-propanediol, sorbitol, or xylitol; the alkane may be pentane, hexane, heptane, octane, nonane, decane, undecane, or dodecane; the cycloalkane may be cyclopentane, cyclohexane, cycloheptane, or cyclooctane; the alkene may be pentene, hexene, heptene, or octane; the amino acid may be aspartic acid, glutamic acid, glycine, lysine, serine, or threonine; the gas may be methane, hydrogen gas, carbon dioxide, or carbon monoxide; the ketone may be acetone; and the organic acid may be acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. In a preferred embodiment, the fermentation product is an alcohol, which may be ethanol, n-butanol, or isobutanol.

In a third aspect, the invention provides a process for producing a fermentation product, the process comprising:
a. pretreatment of lignocellulosic biomass containing cellulose and/or hemicellulose by hot water pretreatment, steam pretreatment, dilute acid pretreatment, wet oxidation, wet explosion pretreatment with organic solvents, biological pretreatment, supercritical CO₂ pretreatment, or ozone pretreatment;
b. preparation of a biomass-enzyme mixture comprising (i) the pretreated lignocellulosic biomass of step (a) and (ii) an enzyme composition comprising cellulase enzymes and/or hemicellulase enzymes;
c. a first saccharification of the biomass-enzyme mixture from step (b) for a sufficient time to achieve hydrolysis of at least about 10% of the cellulose and/or hemicellulose and to produce a suspension of partially-hydrolyzed biomass and hydrolysate liquor;
d. mechanical treatment of the suspension of partially-hydrolyzed biomass produced by step
(c) to produce mechanically-disrupted, partially-hydrolyzed biomass;
e. a second saccharification of the mechanically-disrupted, partially-hydrolyzed biomass produced by step (d) for a sufficient time to achieve hydrolysis of at least about 60% of the cellulose and/or hemicellulose to fermentable sugars;
f. fermenting the fermentable sugars produced in step (e) with one or more fermenting microorganisms to produce the fermentation product; and
g. recovering the fermentation product from the fermentation.

### Enzyme Compositions

The enzyme compositions can comprise any protein that is useful in saccharifying cellulosic material, including biomass, lignocellulose or pretreated lignocellulose

In one aspect, the enzyme composition comprises one or more (several) cellulase enzymes. In another aspect, the enzyme composition comprises or further comprises one or more (several) hemicellulase enzymes. In another aspect, the enzyme composition comprises one or more (several) cellulase enzymes and one or more (several) hemicellulase enzymes. In another aspect, the enzyme composition comprises one or more (several) enzymes selected from the group of cellulase enzymes and hemicellulase enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises a polypeptide having cellulolytic enhancing activity. In another aspect, the enzyme composition comprises an endoglucanase and a polypeptide having cellulolytic enhancing activity. In another aspect, the enzyme composition comprises a cellobiohydrolase and a polypeptide having cellulolytic enhancing activity. In another aspect, the enzyme composition comprises a beta-glucosidase and a polypeptide having cellulolytic enhancing activity. In another aspect, the enzyme composition comprises an endoglucanase and a cellobiohydrolase. In another aspect, the enzyme composition comprises an endoglucanase and a beta-glucosidase. In another aspect, the enzyme composition comprises a cellobiohydrolase and a beta-glucosidase. In another aspect, the enzyme composition comprises an endoglucanase, a cellobiohydrolase, and a polypeptide having cellulolytic enhancing activity. In another aspect, the enzyme composition comprises an endoglucanase, a beta-glucosidase, and a polypeptide having cellulolytic enhancing activity. In another aspect, the enzyme composition comprises a cellobiohydrolase, a beta-glucosidase, and a polypeptide having cellulolytic enhancing activity. In another aspect, the enzyme composition comprises an endoglucanase, a cellobiohydrolase, a beta-glucosidase, and a polypeptide having cellulolytic enhancing activity.

In one aspect, the one or more (e.g., several) cellulase enzymes comprise a commercial cellulase preparation. Examples of commercial cellulase preparations suitable for use in the present invention include, for example, CELLIC® CTec (Novozymes A/S), CELLIC® CTec2 (Novozymes A/S), CELLIC® CTec3 (Novozymes A/S), CELLUCLAST™ (Novozymes A/S), NOVOZYM™ 188 (Novozymes A/S), SPEZYME™ CP (Genencor Int.), ACCELERASE™ TRIO (DuPont), FILTRASE® NL (DSM); METHAPLUS® S/L 100 (DSM), ROHAMENT™ 7069 W (Röhm GmbH), or ALTERNAFUEL® CMAX3™ (Dyadic International, Inc.).

Other useful endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be useful in the present invention are described in EP 495,257, EP 531,315, EP 531,372, WO 89/09259, WO 94/07998, WO 95/24471, WO 96/11262, WO 96/29397, WO 96/034108, WO 97/14804, WO 98/08940, WO 98/012307, WO 98/13465, WO 98/015619, WO 98/015633, WO 98/028411, WO 99/06574, WO 99/10481, WO 99/025846, WO 99/025847, WO 99/031255, WO 2000/009707, WO 2002/050245, WO 2002/0076792, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 4,435,307, U.S. Pat. No. 5,457,046, U.S. Pat. No. 5,648,263, U.S. Pat. No. 5,686,593, U.S. Pat. No. 5,691,178, U.S. Pat. No. 5,763,254, and U.S. Pat. No. 5,776,757.

In another aspect the enzyme composition comprises or further comprises one or more (several) proteins selected from the group consisting of a cellulase, an AA9 polypeptide having cellulolytic enhancing activity, a hemicellulase, an expansin, an esterase, a laccase, a ligninolytic enzyme, a pectinase, a peroxidase, a protease, and a swollenin. In another aspect, the cellulase is preferably one or more (several) enzymes selected from the group consisting of an endoglucanase, a cellobiohydrolase, and a beta-glucosidase. In another aspect, the hemicellulase is one or more (several) enzymes selected from the group consisting of an acetylmannan esterase, an acetyxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase, or any combination thereof.

In another aspect, the enzyme composition comprises or further comprises an acetylmannan esterase. In another aspect, the enzyme composition comprises or further comprises an acetyxylan esterase. In another aspect, the enzyme composition comprises or further comprises an arabinanase (e.g., alpha-L-arabinanase). In another aspect, the enzyme composition comprises or further comprises an arabinofuranosidase (e.g., alpha-L-arabinofuranosidase). In another aspect, the enzyme composition comprises or further comprises a coumaric acid esterase. In another aspect, the enzyme composition comprises or further comprises a feruloyl esterase. In another aspect, the enzyme composition comprises or further comprises a galactosidase (e.g., alpha-galactosidase and/or beta-galactosidase). In another aspect, the enzyme composition comprises or further comprises a glucuronidase (e.g., alpha-D-glucuronidase). In another aspect, the enzyme composition comprises or further comprises a glucuronoyl esterase. In another aspect, the enzyme composition comprises or further comprises a mannanase. In another aspect, the enzyme composition comprises or further comprises a mannosidase (e.g., beta-mannosidase). In another aspect, the enzyme composition comprises or further comprises a xylanase, which may be a Family 10 xylanase. In another aspect, the enzyme composition comprises or further comprises a xylosidase (e.g., beta-xylosidase). In another aspect, the enzyme composition comprises or further comprises an expansin.

In another aspect, the enzyme composition comprises or further comprises an esterase. In another aspect, the enzyme composition comprises or further comprises a laccase. In another aspect, the enzyme composition comprises or further comprises a ligninolytic enzyme, such as a manganese peroxidase or a lignin peroxidase. In another aspect, the ligninolytic enzyme is a H₂O₂-producing enzyme. In another aspect, the enzyme composition comprises or further comprises a pectinase. In another aspect, the enzyme composition comprises or further comprises a peroxidase. In another aspect, the enzyme composition comprises or further comprises a protease. In another aspect, the enzyme composition comprises or further comprises a swollenin.

In one aspect, the one or more (e.g., several) hemicellulase enzymes comprise a commercial hemicellulase preparation. Examples of commercial hemicellulase enzymes suitable for use in the present invention include, for example, SHEARZYME™ (Novozymes A/S), CELLIC® HTec (Novozymes A/S), CELLIC® HTec2 (Novozymes A/S), CELLIC® HTec3 (Novozymes A/S), VISCOZYME® (Novozymes A/S), ULTRAFLO® (Novozymes A/S), PULPZYME® HC (Novozymes NS), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK), ALTERNA FUEL 100P (Dyadic), and ALTERNA FUEL 200P (Dyadic).

One or more (several) components of the enzyme composition may be wild-type proteins, recombinant proteins, or a combination of wild-type proteins and recombinant proteins. For example, one or more (several) components may be native proteins of a cell, which is used as a host cell to express recombinantly one or more (several) other components of the enzyme composition. One or more (several) components of the enzyme composition may be produced as monocomponents, which are then combined to form the enzyme composition. The enzyme composition may be a combination of multicomponent and monocomponent protein preparations.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host may be a heterologous host (enzyme is foreign to host), but the host may, under certain conditions, also be a homologous host (enzyme is native to host). Monocomponent cellulolytic proteins may also be prepared by purifying such a protein from a fermentation broth.

The enzymes used in the processes of the present invention may be in any form suitable for use, such as, for example, a crude fermentation broth with or without cells removed, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The polypeptides having cellulase enzyme activity or hemicellulase enzyme activity as well as other proteins/polypeptides useful in the degradation of the biomass material, e.g., AA9 polypeptides (collectively hereinafter “polypeptides having enzyme activity”) can be derived or obtained from any suitable origin, including, bacterial, fungal, yeast, plant, or mammalian origin. The term “obtained” means herein that the enzyme may have been isolated from an organism that naturally produces the enzyme as a native enzyme. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (several) amino acids that are deleted, inserted and/or substituted, i.e., a recombinantly produced enzyme that is a mutant and/or a fragment of a native amino acid sequence or an enzyme produced by nucleic acid sequence mutagenesis processes known in the art. Encompassed within the meaning of a native enzyme are natural variants and within the meaning of a foreign enzyme are variants obtained recombinantly, such as by site-directed mutagenesis or random mutagenesis.

The polypeptide having enzyme activity may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity. In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity. In another aspect, the polypeptide is a Streptococcus equisimilis, Streptococcus pyogenes, Streptococcus uberis, or Streptococcus equi subsp. Zooepidemicus polypeptide having enzyme activity. In another preferred aspect, the polypeptide is a Streptomyces achromogenes, Streptomyces avermitilis, Streptomyces coelicolor, Streptomyces griseus, or Streptomyces lividans polypeptide having enzyme activity.

The polypeptide having enzyme activity may also be a yeast polypeptide such as a Candida, Kluyveromyces, Pichia, Saccharomyces, Schizosaccharomyces, or Yarrowia polypeptide having enzyme activity. In one aspect, the polypeptide may be a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

The polypeptide having enzyme activity may also be a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity. In one aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulfureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of the polypeptides having enzyme activity may also be used.

The polypeptides having enzyme activity used in the processes of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

### Pretreated Lignocellulosic Biomass

In the process of the present invention, the biomass-enzyme mixture comprises pre-treated lignocellulosic biomass (or lignocellulose). Any pretreatment process known in the art may be used to disrupt plant cell wall components of the biomass (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: 10-18; Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40). The biomass may also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion or expansion (sometimes referred to as ammonia freeze explosion or “AFEX”), organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

Steam Pretreatment.

In steam pretreatment, lignocellulosic biomass is heated to disrupt the plant cell wall components, including lignin, hemicellulose, and cellulose to make the cellulose and other fractions, e.g., hemicellulose, accessible to enzymes. The lignocellulosic biomass is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on the temperature and addition of a chemical catalyst. Steam pretreatment allows for relatively high solids loadings, so that the lignocellulosic biomass is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 2002/0164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment:

The term “chemical pretreatment” refers to any chemical pretreatment that promotes the separation and/or release of cellulose, hemicellulose, and/or lignin. Such a pretreatment may convert crystalline cellulose to amorphous cellulose. Examples of suitable chemical pretreatment processes include, for example, dilute acid pretreatment, lime pretreatment, wet oxidation, ammonia fiber/freeze expansion (AFEX), ammonia percolation (APR), ionic liquid, and organosolv pretreatments.

A catalyst such as H₂SO₄ or SO₂ (typically 0.3 to 5% w/w) is often added prior to steam pretreatment, which decreases the time and temperature, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technology 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze expansion (AFEX) pretreatment.

Lime pretreatment is performed with calcium oxide or calcium hydroxide at temperatures of 85-150° C. and residence times from 1 hour to several days (Wyman et al., 2005, Bioresource Technology 96: 1959-1966; Mosier et al., 2005, Bioresource Technology 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technology 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber expansion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technology 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. and Biotechnol. 105-108: 69-85, and Mosier et al., 2005, Bioresource Technology 96: 673-686, and U.S. Published Application 2002/0164730.

In one aspect, the pretreated lignocellulosic biomass is produced by chemical pretreatment. In another aspect, the chemical pretreatment is as a hot water pretreatment, steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation.

In another aspect, the chemical pretreatment is a hot water pretreatment, steam pretreatment or dilute acid pretreatment.

In another aspect, the chemical pretreatment is a hot water pretreatment. Hot water pretreatment may be conducted at a temperature in the range of preferably 140-200° C., e.g., 165-190° C., for periods ranging from 1 to 60 minutes.

In some aspects, the cellulosic material is present during pretreatment in amounts preferably between 10-80 wt. %, e.g., 20-70 wt. % or 30-60 wt. %, such as around 40 wt. %. The pretreated cellulosic material can be unwashed or washed using any method known in the art, e.g., washed with water.

In another aspect, the chemical pretreatment is a dilute or mild acid pretreatment, for example, a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt. % acid, e.g., 0.05 to 5 wt. % acid or 0.1 to 2 wt. % acid. The acid is contacted with the cellulosic material and held at a temperature in the range of preferably 140-200° C., e.g., 165-190° C., for periods ranging from 1 to 60 minutes.

In another aspect, pretreatment takes place in an aqueous slurry. In preferred aspects, the cellulosic material is present during pretreatment in amounts preferably between 10-80 wt. %, e.g., 20-70 wt. % or 30-60 wt. %, such as around 40 wt. %. The pretreated cellulosic material can be unwashed or washed using any method known in the art, e.g., washed with water.

Mechanical Pretreatment or Physical Pretreatment:

The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperature in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, the cellulosic material may be subjected to physical (mechanical) or chemical pretreatment, or any combination thereof, to promote the separation and/or release of cellulose, hemicellulose, and/or lignin.

Biological Pretreatment:

The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Enz. Microb. Tech. 18: 312-331; and Vallander and Eriksson, 1990, Adv. Biochem. Eng./Biotechnol. 42: 63-95).

### Biomass-Enzyme Mixtures

The first step of the process of the present invention is a preparation of a biomass-enzyme mixture of (i) lignocellulosic biomass pretreated as described above and (ii) an enzyme composition comprising cellulase enzymes and/or hemicellulase enzymes, as described above.

The optimum amounts of the enzyme composition depend on several factors including, but not limited to, the mixture of cellulase enzymes and/or hemicellulase enzymes, the lignocellulosic biomass, the concentration of biomass, the pretreatment(s) of the biomass, temperature, time, pH, and inclusion of a fermenting organism (e.g., for Simultaneous Saccharification and Fermentation).

In one aspect, the amount of cellulase enzymes and/or hemicellulase enzymes to the pretreated lignocellulosic biomass is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the pretreated lignocellulosic biomass.

The total solids (TS) during treatment with an enzymatic composition is preferably about 1% to about 50%, e.g., about 2% to about 40%, about 2% to about 35%, about 3% to about 30%, about 3% to about 25%, about 4% to about 20%, or about 5% to about 10%.

### Saccharification

In the saccharification step, also known as hydrolysis, the pretreated lignocellulosic biomass is hydrolyzed to break down cellulose and/or hemicellulose to fermentable sugars, such as glucose, cellobiose, xylose, xylulose, arabinose, mannose, galactose, and/or soluble oligosaccharides. The hydrolysis is performed enzymatically by an enzyme composition. Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The process of the present invention involves at least two saccharification steps. The first saccharification step occurs subsequent to the preparation of the above described biomass-enzyme mixture. The second and subsequent saccharification steps occur after a mechanical treatment of partially hydrolyzed biomass.

The pH during the saccharification steps should fall into a range at which the activity of the enzyme(s) being used is optimal. For example, in the case of cellulases or hemicellulases, the saccharification may be performed at a pH of about 3 to about 8, e.g., about 3 to about 7.5, about 3.5 to about 7, about 4 to about 6.5, about 4.5 to about 6.5, about 4.5 to about 6.0, about 5 and about 6.0, or about 5 to about 5.5. In one embodiment, the pH during enzymatic pretreatment is at about pH 5.

The temperature during saccharification steps should be at or near the optimum for the enzyme(s) being used. In the case of cellulases or hemicellulases, the temperature is preferably about 20° C. to about 70° C., e.g., about 25° C. to about 65° C., about 30° C. to about 65° C., about 35° C. to about 65° C., about 40° C. to about 60° C., about 45° C. to about 55° C., or about 45° C. to about 50° C.

In the first saccharification step, the biomass-enzyme mixture is incubated for a sufficient time to achieve hydrolysis of at least about 10% of the cellulose and/or hemicellulose in the pretreated lignocellulosic biomass, resulting in the production of partially-hydrolyzed biomass. In some aspects, the incubation time of the first saccharification step is sufficient to achieved hydrolysis of from about 10% to about 60%, e.g., from about 10% to about 55%, e.g., from about 10% to about 50%, e.g., from about 10% to about 45%, e.g., from about 10% to about 40%, e.g., from about 10% to about 35%, e.g., from about 10% to about 30%, e.g., from about 10% to about 25%, e.g., from about 10% to about 20%, e.g., from about 10% to about 15% of the cellulose and/or hemicellulose in the pretreated lignocellulosic biomass. The incubation time of the first saccharification step can vary depending on the dose of the enzyme composition. At a preferable dose, the incubation time may range from 1 to 96 hours, e.g., 2 to 96 hours, 6 to 96 hours, 12 to 96 hours, 24 to 96 hours, 6 to 72 hours, 12 to 72 hours, or 24 to 72 hours, or any incubation time therebetween. However, any appropriate incubation time can be used and is easily determined by one skilled in the art.

In the second, and optional subsequent, saccharification step(s), the biomass-enzyme mixture is incubated for a sufficient time to achieve hydrolysis of at least about 60% of the cellulose and/or hemicellulose in the pretreated lignocellulosic biomass. In some aspects, the incubation time of the second, and optional subsequent, saccharification step(s), is sufficient to achieve hydrolysis of about 60% to about 100%, e.g., about 60% to about 95%, e.g., about 60% to about 90%, e.g., about 60% to about 85%, e.g., about 60% to about 80%, e.g., about 60% to about 75%, e.g., about 60% to about 70%, e.g., about 65% to about 100%, e.g., about 70% to about 100%, e.g., about 75% to about 100%, e.g., about 80% to about 100%, e.g., about 85% to about 100%, e.g., about 90% to about 100%, or about 95% to about 100%, of the cellulose and/or hemicellulose in the pretreated lignocellulosic biomass. The incubation time of the second, and optional subsequent, saccharification step(s), can vary depending on the dose of the enzyme composition. At a preferable dose, the incubation time may range from 1 to 96 hours, e.g., 2 to 96 hours, 6 to 96 hours, 12 to 96 hours, 24 to 96 hours, 6 to 72 hours, 12 to 72 hours, or 24 to 72 hours, or any incubation time therebetween. However, any appropriate incubation time can be used and is easily determined by one skilled in the art.

A saccharification step may further comprise inactivating the enzyme composition and/or filtering the partially-hydrolyzed biomass. In one aspect, a saccharification step further comprises inactivating the enzyme composition. In another aspect, a saccharification step further comprises filtering the partially-hydrolyzed biomass. In another aspect, a saccharification step further comprises inactivating the enzyme composition and filtering the partially-hydrolyzed biomass.

The inactivation of the enzyme composition may be performed at any temperature and period of time suitable for inactivation the enzyme composition. In one aspect, the temperature is at least 80° C., e.g., at least 85° C., at least 90° C., at least 95° C., or at least 100° C. for at least 10 minutes, e.g., at least 20 minutes, at least 30 minutes, at least 45 minutes, or at least 60 minutes. In another aspect, the temperature is about 85° C. for about 30 minutes.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); separate hydrolysis and co-fermentation (SHCF); hybrid hydrolysis and co-fermentation (HHCF); and direct microbial conversion (DMC). SHF uses separate process steps to first enzymatically hydrolyze cellulosic material to fermentable sugars, e.g., glucose, cellobiose, cellotriose, and pentose sugars, and then ferment the fermentable sugars to ethanol. In SSF, the enzymatic hydrolysis of cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the cofermentation of multiple sugars (Sheehan, J., and Himmel, M., 1999, Enzymes, energy and the environment: A strategic perspective on the U.S. Department of Energy's research and development activities for bioethanol, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (several) steps where the same organism is used to produce the enzymes for conversion of the cellulosic material to fermentable sugars and to convert the fermentable sugars into a final product (Lynd, L. R., Weimer, P. J., van Zyl, W. H., and Pretorius, I. S., 2002, Microbial cellulose utilization: Fundamentals and biotechnology, Microbiol. Mol. Biol. Reviews 66: 506-577). It is understood herein that any method known in the art comprising pretreatment, enzymatic hydrolysis (saccharification), fermentation, or a combination thereof, can be used in the practicing the processes of the present invention.

A conventional apparatus for saccharification can include a fed-batch stirred reactor, a batch stirred reactor, a continuous flow stirred reactor with ultrafiltration, and/or a continuous plug-flow column reactor (Fernanda de Castilhos Corazza, Fl\dot{a}vio Faria de Moraes, Gisella Maria Zanin and Ivo Neitzel, 2003, Optimal control in fed-batch reactor for the cellobiose hydrolysis, Acta Scientiarum. Technology 25: 33-38; Gusakov, A. V., and Sinitsyn, A. P., 1985, Kinetics of the enzymatic hydrolysis of cellulose: 1. A mathematical model for a batch reactor process, Enz. Microb. Technol. 7: 346-352), an attrition reactor (Ryu, S. K., and Lee, J. M., 1983, Bioconversion of waste cellulose by using an attrition bioreactor, Biotechnol. Bioeng. 25: 53-65), or a reactor with intensive stirring induced by an electromagnetic field (Gusakov, A. V., Sinitsyn, A. P., Davydkin, I. Y., Davydkin, V. Y., Protas, O. V., 1996, Enhancement of enzymatic cellulose hydrolysis using a novel type of bioreactor with intensive stirring induced by electromagnetic field, Appl. Biochem. Biotechnol. 56: 141-153). Additional reactor types include: fluidized bed, upflow blanket, immobilized, and extruder type reactors for hydrolysis and/or fermentation.

### Mechanical Treatments

In the processes of the present invention, mechanical treatment of the partially-hydrolyzed biomass makes the cellulose and/or hemicellulose more susceptible to hydrolysis by cellulase and/or hemicellulase enzymes. The processes of the present invention provide a means of increasing the kinetics of saccharification of a pretreated lignocellulosic biomass and making the cellulose and/or hemicellulose in the biomass more amenable to the action of the cellulase and/or hemicellulase enzymes. Advantages of the processes of the present invention include lowered dose of cellulase and/or hemicellulase enzymes during hydrolysis, increased yield of fermentable sugar, and faster hydrolysis rates.

In the processes of the present invention, the partially-hydrolyzed biomass produced in the first, or second, saccharification step is subjected to a mechanical treatment to produce mechanically disrupted, partially-hydrolyzed biomass. The term “mechanical treatment” refers to various types of refining, milling (e.g., dry milling, wet milling, or vibratory ball milling), crushing, grinding, shredding, extrusion, beating, or combinations thereof. The mechanical treatment uses hydraulic and/or mechanical forces to generate a shearing action on the partially-hydrolyzed biomass. This has the effect of increasing surface area due to fibrillation, shortening, and bruising while also increasing flexibility and hydration, and also opening up the structure for improved saccharification.

The mechanical treatment can be performed with any device known in the art including, but not limited to, extruders, conical and disk refiners, hydrapulpers, deflakers, beaters, and/or any other devices designed to generate shearing forces that may alter or disrupt the cellulosic material.

In one aspect of the present invention, the mechanical treatment is refining. In some embodiments, the refining is conducted as to provide a refining energy of about 50 to about 500 kWh per dry tonne of biomass, e.g, 50 to 450 kWh per dry tonne of biomass, e.g, 50 to 400 kWh per dry tonne of biomass, e.g, 50 to 350 kWh per dry tonne of biomass, e.g, 50 to 300 kWh per dry tonne of biomass, e.g, 100 to 400 kWh per dry tonne of biomass, e.g, 100 to 350 kWh per dry tonne of biomass, or e.g, 100 to 300 kWh per dry tonne of biomass.

The disk refining may be conducted with a laboratory PFI refiner. In one aspect, the partially-hydrolyzed biomass is refined for 3,000 to 20,000 counts, e.g., for 5,000 to 20,000 counts, e.g., for 5,000 to 15,000 counts, e.g., for 5,000 to 10,000 counts, e.g., for 3,000 to 10,000 counts, e.g., for 3,000 to 5,000 counts

The consistency of the partially-hydrolyzed biomass prior to the mechanical treatment may be in the range of 1% to 40%, e.g., 1% to 35%, e.g., 1% to 30%, e.g., 1% to 25%, e.g., 1% to 20%, e.g., 1% to 15%, e.g., 1% to 10%, e.g., 1% to 8%, e.g., 1% to 6%, e.g., 1% to 4%, e.g., 1% to 2%, e.g., 5% to 40%, e.g., 10% to 40%, e.g., 15% to 40%, e.g., 20% to 40%, e.g., 30% to 40%, e.g., 5% to 20%, e.g., from 5% to 15%, e.g., 5% to 10%.

Depending upon the choice of mechanical treatment method, the equipment, the initial concentration of biomass in the biomass-enzyme mixture, and the extent of hydrolysis in the saccharification step preceding the mechanical treatment, the consistency of the partially-treated biomass may need to be increased. In one aspect, the processes of the present invention further comprise separating the partially-hydrolyzed biomass from enzyme- and sugar-containing liquor after a saccharification step. Separation of the partially-hydrolyzed biomass from the liquor may be performed following saccharification to increase the total solids to about 20% to about 45%, e.g., about 20%, about 25%, about 30%, about 35%, about 40%, or about 45%.

Separation of the partially-hydrolyzed biomass from the liquor may be carried out using any method known in the art including, but not limited to, filtration, pressure filtration, vacuum filtration, gravity settling, decantation, centrifugation, belt press, or screw press. The filtration of the partially-hydrolyzed biomass can be accomplished using any method for solid liquid separation known in the art. Such non-limiting examples of solid liquid separation methods include rotary vacuum washers, rotary pressure washers, diffusion washers, horizontal belt washers, screw presses, wash presses, pulp screens, centrifugal gravity screens, decanters, and centrifuges. All of these methods also allow for efficient recycling of enzyme containing liquor back to the saccharification step with additional enzyme being added as necessary. See, for example, Kraft Pulping. “A Compilation of Notes” Chapter 6, Pulp Processing, pp 115-133, ISBN#0-89852-322-2, TAPPI Press, 1993; Smook, G.A. Handbook for Pulp & Paper Technologists Chapter 9, Processing of Pulps, pp. 89-112, ISBN#0-919893-00-7, TAPPI Press, 1982.

In one aspect, no additional enzyme composition is added to the mechanically disrupted, partially-hydrolyzed biomass prior to the second (or subsequent) saccharification steps.

In another aspect, an additional amount of the enzyme composition can be added to the mechanically disrupted, partially-hydrolyzed biomass according to the amounts described herein to further increase the rate or extent of conversion of the cellulose and/or hemicellulose.

### Fermentation

The fermentable sugars obtained from the hydrolyzed lignocellulosic biomass can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the lignocellulosic biomass as a result of the pretreatment, saccharification and mechanical treatment steps, are fermented to a product, e.g., ethanol, by a fermenting organism, such as yeast. Hydrolysis (saccharification) and fermentation can be separate or simultaneous.

Any suitable hydrolyzed lignocellulosic biomass can be used in the fermentation step in practicing the present invention. The material is generally selected based on economics, i.e., costs per equivalent sugar potential, and recalcitrance to enzymatic conversion.

The term “fermentation medium” is understood herein to refer to a medium before the fermenting microorganism(s) is(are) added, such as, a medium resulting from a saccharification process, as well as a medium used in a simultaneous saccharification and fermentation process (SSF). “Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Yeast include strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Xylose fermenting yeast include strains of Candida, preferably C. sheatae or C. sonorensis; and strains of Pichia, e.g., P. stipitis, such as P. stipitis CBS 5773. Pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, 1996, supra).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K. lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

Commercially available yeast suitable for ethanol production include, e.g., BIOFERM™ AFT and XR (NABC—North American Bioproducts Corporation, GA, USA), ETHANOL RED™ yeast (Fermentis/Lesaffre, USA), FALI™ (Fleischmann's Yeast, USA), FERMIOL™ (DSM Specialties), GERT STRAND™ (Gert Strand AB, Sweden), and SUPERSTART™ and THERMOSACC™ fresh yeast (Ethanol Technology, WI, USA).

In an aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (co-fermentation) (Chen and Ho, 1993, Appl. Biochem. Biotechnol. 39-40: 135-147; Ho et al., 1998, Appl. Environ. Microbiol. 64: 1852-1859; Kotter and Ciriacy, 1993, Appl. Microbiol. Biotechnol. 38: 776-783; Walfridsson et al., 1995, Appl. Environ. Microbiol. 61: 4184-4190; Kuyper et al., 2004, FEMS Yeast Research 4: 655-664; Beall et al., 1991, Biotech. Bioeng. 38: 296-303; Ingram et al., 1998, Biotechnol. Bioeng. 58: 204-214; Zhang et al., 1995, Science 267: 240-243; Deanda et al., 1996, Appl. Environ. Microbiol. 62: 4465-4470; WO 03/062430).

It is well known in the art that the organisms described above can also be used to produce other substances, as described herein.

The fermenting microorganism is typically added to the degraded cellulosic material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded cellulosic material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

### Fermentation Products

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In one aspect, the fermentation product is an alcohol. It will be understood that the term “alcohol” encompasses a substance that contains one or more hydroxyl moieties. The alcohol can be, but is not limited to, n-butanol, isobutanol, ethanol, methanol, arabinitol, butanediol, ethylene glycol, glycerin, glycerol, 1,3-propanediol, sorbitol, xylitol. See, for example, Gong et al., 1999, Ethanol production from renewable resources, in Advances in Biochemical Engineering/Biotechnology, Scheper, T., ed., Springer-Verlag Berlin Heidelberg, Germany, 65: 207-241; Silveira and Jonas, 2002, Appl. Microbiol. Biotechnol. 59: 400-408; Nigam and Singh, 1995, Process Biochemistry 30(2): 117-124; Ezeji et al., 2003, World Journal of Microbiology and Biotechnology 19(6): 595-603.

In another aspect, the fermentation product is an alkane. The alkane may be an unbranched or a branched alkane. The alkane can be, but is not limited to, pentane, hexane, heptane, octane, nonane, decane, undecane, or dodecane.

In another aspect, the fermentation product is a cycloalkane. The cycloalkane can be, but is not limited to, cyclopentane, cyclohexane, cycloheptane, or cyclooctane.

In another preferred aspect, the fermentation product is an alkene. The alkene may be an unbranched or a branched alkene. The alkene can be, but is not limited to, pentene, hexene, heptene, or octene.

In another aspect, the fermentation product is an amino acid. The organic acid can be, but is not limited to, aspartic acid, glutamic acid, glycine, lysine, serine, or threonine. See, for example, Richard and Margaritis, 2004, Biotechnology and Bioengineering 87(4): 501-515.

In another aspect, the fermentation product is a gas. The gas can be, but is not limited to, methane, H₂, CO₂, or CO. See, for example, Kataoka et al., 1997, Water Science and Technology 36(6-7): 41-47; and Gunaseelan, 1997, Biomass and Bioenergy 13(1-2): 83-114.

In another aspect, the fermentation product is isoprene.

In another aspect, the fermentation product is a ketone. It will be understood that the term “ketone” encompasses a substance that contains one or more ketone moieties. The ketone can be, but is not limited to, acetone.

In another aspect, the fermentation product is an organic acid. The organic acid can be, but is not limited to, acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, or xylonic acid. See, for example, Chen and Lee, 1997, Appl. Biochem. Biotechnol. 63-65: 435-448.

In another aspect, the fermentation product is polyketide.

Recovery.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### EXAMPLES

### Example 1

### Substrate Preparation

200 g (dry wt) of corn stover was brought to a solids content of 40% using tap water and then pretreated using a Parr reactor equipped with direct steam injection. The temperature was adjusted to 180° C. and held for 20 minutes at a constant stirring rate of 100 rpm. After the pretreatment time was completed, the material was steam exploded into a cyclone and collected for washing. This is similar to the method of Balleros et al. (2006, Appl. Biochem. Biotechnol. 129-132, p. 496-508). The washing was performed by bringing the solids to a volume of 2 liters and then filtering through a fine mesh nylon bag; this was performed 3 times. Following washing and squeezing, the substrate was digested with concentrated acid for composition analysis of carbohydrates and lignin according to the NREL Laboratory Analytical Procedure NREL/TP-510-42618 for Determination of Structural Carbohydrates and Lignin in Biomass (issued April 2008 and revised August 2012). The composition of the pretreated corn stover, in % of dry wt, was determined to be 50.8% glucan (cellulose+beta-glucan), 17.7% xylan, 0.8% galactan, 2.0% arabinan, and 22.4% lignin.

### Example 2

### Saccharification with Refinining

### Example 2.1

### First Saccharification

Following washing, 542.8 g of washed pretreated corn stover (with a total solids content of 31.5%) was placed into each of four 2 L Erlenmeyer flasks. Following the addition of penicillin, 1M acetate buffer pH 5.0, and enzyme solution, deionized water was added to adjust the total solids to 10% in each flask as shown in Table 1. The enzyme solution consisted of an 80/20 blend, in relation to protein concentration, of CELLIC® CTec3 (Novozymes A/S) to CELLIC® HTec3 (Novozymes A/S); the total protein concentration in each flask was 5 mg/ml. The enzyme dosage was based upon the insoluble solids in each flask and was targeted to be 2 mg protein/g insoluble solids.

TABLE 1
Preparation of biomass-enzyme mixtures prior to saccharification and refining

| | Washed | | | | 1M pH 5 | Washed | De-ionized | 1M pH 5 | 1 g/L | Enzyme | |
|---|---|---|---|---|---|---|---|---|---|---|---|
| | substrate | Enzyme | IS in | Hydrolysis | Acetate buff- | substrate | water | Acetate buff- | penicillin | solution | pH |
| Hydrolysis | IS | dose | hydrolysis | slurry wt | er density | loaded | loaded | er added | added | add [symbol] @0 h | after |
| assay # | (g/g) | (g-prot/IS) | (g/g) | ( [symbol] | (g/mL) | (g) | (g) | (mL) | (m/L) | (mL) | 72 h |
| 1 | 31.5% | 2.00 | 10% | 881.9 | 1.135 | 280.0 | 514.3 | 44.1 | 2.20 | 35.27 | 4.73 |
| 2 | 31.5% | 2.00 | 10% | 881.9 | 1.135 | 280.0 | 514.3 | 44.1 | 2.20 | 35.27 | 4.73 |
| 3 | 31.5% | 2.00 | 10% | 881.9 | 1.135 | 280.0 | 514.3 | 44.1 | 2.20 | 35.27 | 4.73 |
| 4 | 31.5% | 2.00 | 10% | 881.9 | 1.135 | 280.0 | 514.3 | 44.1 | 2.20 | 35.27 | 4.73 |

[symbol]

indicates data missing or illegible when filed

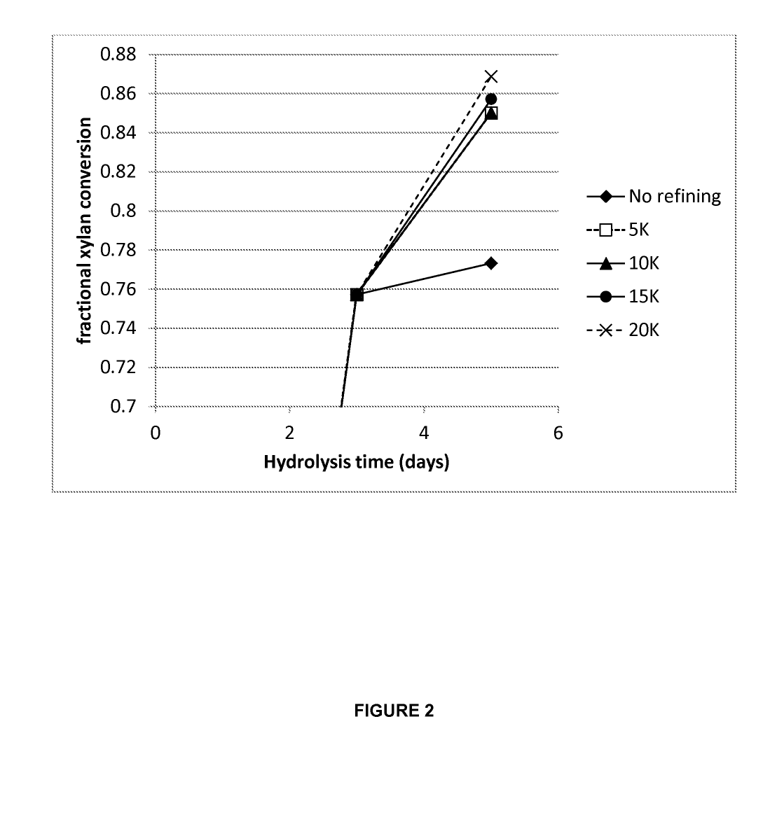

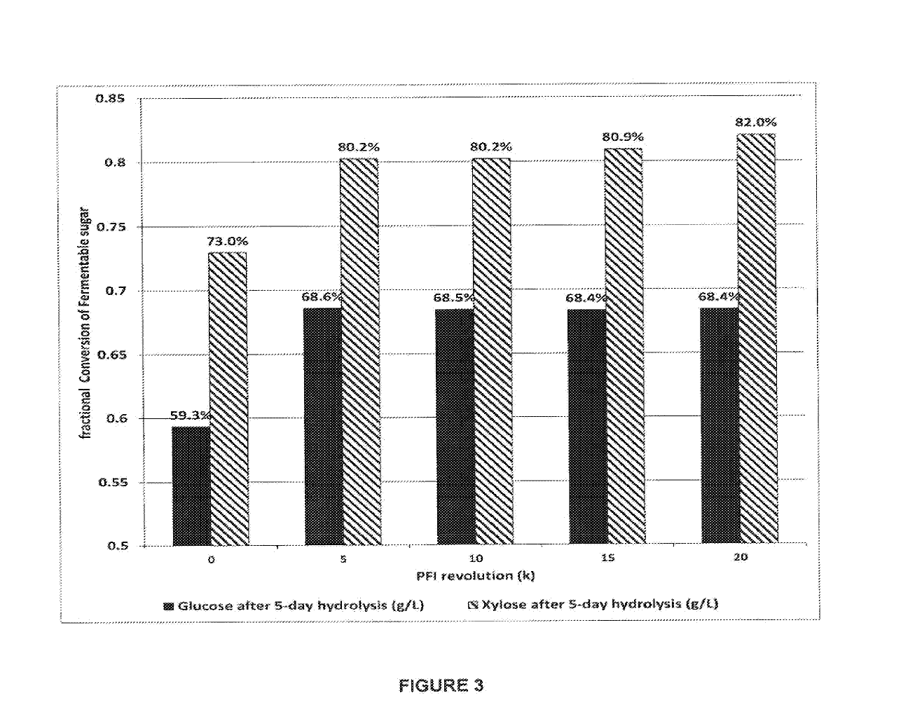

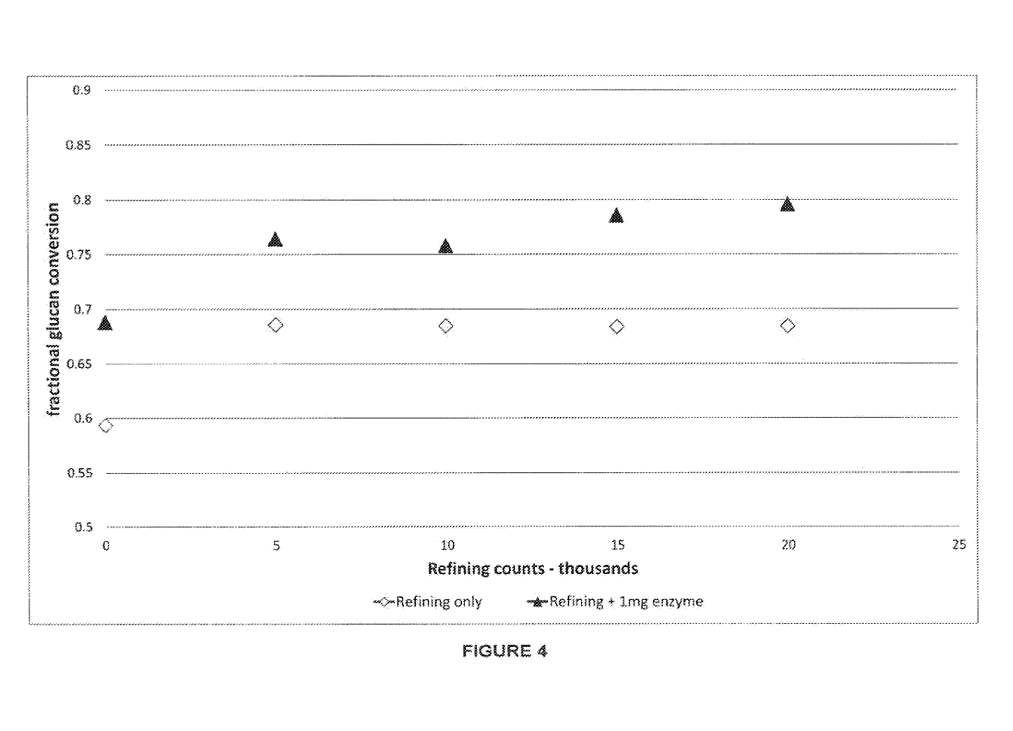

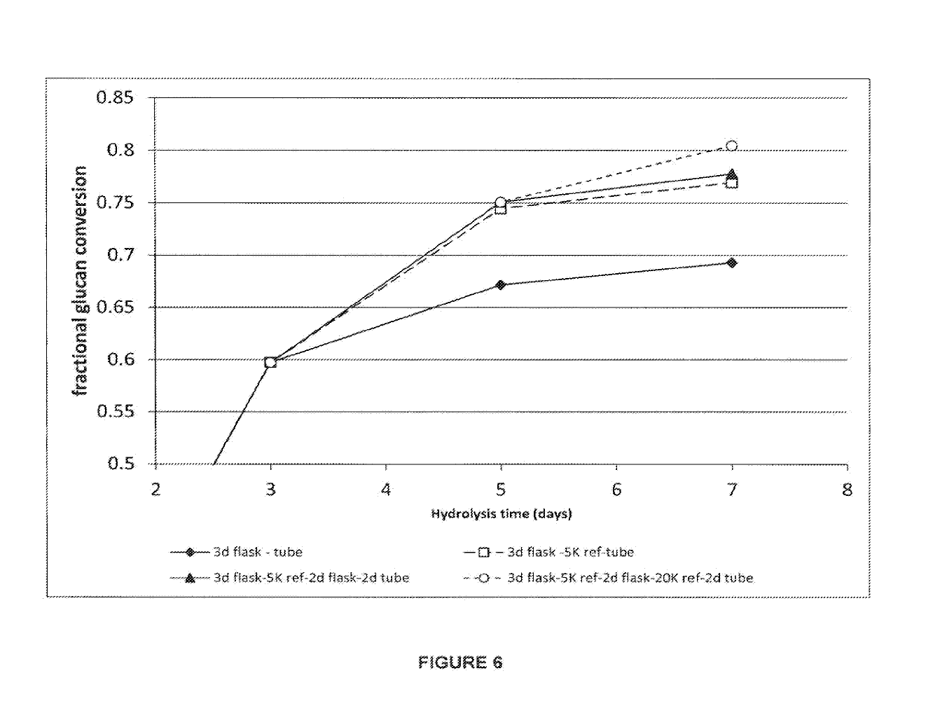

Samples were collected at the time points shown in FIGS. 2-7 and the fractional conversion of glucan (cellulose plus beta-glucan) and xylan into soluble sugars was determined according to the NREL Laboratory Analytical Procedure NREL/TP-510-42623 for Determination of Sugars, Byproducts and Degradation Products in Liquid Fraction Process Samples (issued December 2006 and revised January 2008). Sugar concentrations were measured using an Agilent 1200 HPLC system equipped with a RID detector maintained at 50° C. and a Bio-Rad Aminex HPX-87H cation exchange maintained at 65° C., using a 5 mM sulfuric acid as a mobile phase with a flow rate or 0.6 mL/min.

Hydrolysis was carried out for 72 hours (3 days) after which the hydrolyzed slurry was placed into 500 mL polycarbonate centrifuge bottles and centrifuged for 15 minutes at 3000×g. This provided the solid liquid separation necessary for the solids to be passed into the first refining stage.

### 2.2 Mechanical Disruption:

Refining was carried out in accordance with TAPPI method T248 “Laboratory beating of Pulp (PFI Mill Method)”. HPLC samples were taken of the supernatant for sugar analysis as described above. The resulting solids were refined for 0, 5, 10, 15, and 20K counts in a laboratory PFI.

### 2.3 Second Saccharification and Mechanical Disruption

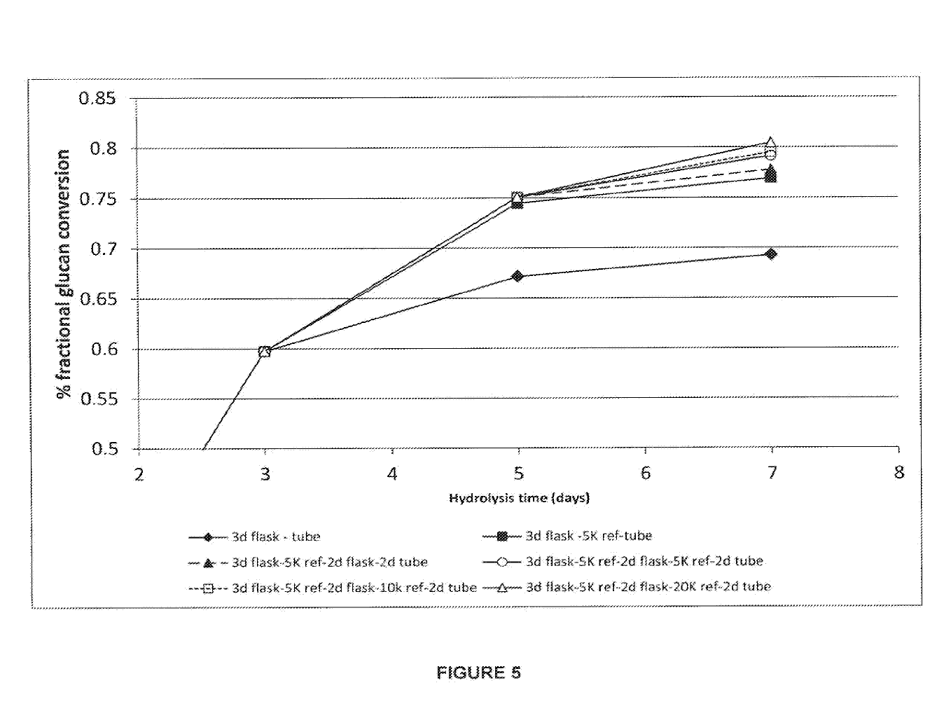

The material from each refining run was then recombined with its supernatant counterpart and allowed to continue hydrolysis for an additional 48 hours after which the centrifugation solids/liquids separation step was repeated and HPLC samples were taken and a second refining step was performed. To half of the samples an additional enzyme—an 80/20 blend of CELLIC® CTec3 (Novozymes A/S)/CELLIC® HTec3 (Novozymes A/S)—was added at a dosage of 1 mg protein/g insoluble solids (FIG. 5).

### Embodiments

The present invention is further described by the following numbered paragraphs:
[1] A process for producing fermentable sugars from biomass, comprising:
   a. preparation of a biomass-enzyme mixture comprising (i) pretreated lignocellulosic biomass containing cellulose and/or hemicellulose and (ii) an enzyme composition comprising cellulase and/or hemicellulase enzymes;
   b. a first saccharification comprising incubation of the biomass-enzyme mixture from step (a) for a sufficient time to achieve hydrolysis of at least about 10% of the cellulose and/or hemicellulose and produce partially-hydrolyzed biomass and hydrolysate liquor;
   c. mechanical treatment of the partially-hydrolyzed biomass produced by step (b) to produce mechanically-disrupted, partially-hydrolyzed biomass; and
   d. a second saccharification comprising incubation of the mechanically-disrupted, partially-hydrolyzed biomass produced by step (c) for a sufficient time to achieve hydrolysis of at least about 60% to 100% of the cellulose and/or hemicellulose present in the pretreated lignocellulosic biomass to fermentable sugars.
[2] A process for producing a fermentation product, comprising
   a. preparation of a biomass-enzyme mixture comprising (i) pretreated lignocellulosic biomass containing cellulose and/or hemicellulose and (ii) an enzyme composition comprising cellulase enzymes and/or hemicellulase enzymes;
   b. a first saccharification of the biomass-enzyme mixture from step (a) for a sufficient time to achieve hydrolysis of at least about 10% the cellulose and/or hemicellulose and produce partially-hydrolyzed biomass and hydrolysate liquor;
   c. mechanical treatment of the partially-hydrolyzed biomass produced by step (b) to produce mechanically-disrupted, partially-hydrolyzed biomass;
   d. a second saccharification of the mechanically-disrupted, partially-hydrolyzed biomass produced by step (c) for a sufficient time to achieve hydrolysis of at least about 60% of the cellulose and/or hemicellulose to fermentable sugars present in the pretreated lignocellulosic biomass;
   e. fermenting the fermentable sugars produced in step (d) with one or more fermenting microorganisms to produce the fermentation product; and
   f. recovering the fermentation product from the fermentation.
[3] The process of paragraph [1] or [2], wherein the second saccharification of step (d) is conducted without an additional dose of cellulase and/or hemicellulase enzymes.
[4] The process of paragraph [1] or [2], wherein the second saccharification of step (d) is conducted with an additional dose of cellulase and/or hemicellulase enzymes.
[5] The process of any one of paragraphs [1] to [3], wherein the mechanical treatment is selected from the group consisting of refining, milling, crushing, grinding, shredding, extrusion, beating, or combinations thereof.
[6] The process of any one of paragraphs [1] to [3], wherein the mechanical treatment is refining.
[7] The process of paragraph [6], wherein the refining is conducted so as to provide a refining energy of 50 to 500 kWh per dry tonne of biomass, 50 to 450 kWh per dry tonne of biomass, to 400 kWh per dry tonne of biomass, 50 to 350 kWh per dry tonne of biomass, 50 to 300 kWh per dry tonne of biomass, 100 to 400 kWh per dry tonne of biomass, 100 to 350 kWh per dry tonne of biomass, or 100 to 300 kWh per dry tonne of biomass.
[8] The process of any one of paragraphs [1] to [3], wherein the mechanical treatment is milling.
[9] The process of any one of paragraphs [1] to [3], wherein the mechanical treatment is crushing or grinding.
[10] The process of any one of paragraphs [1] to [3], wherein the mechanical treatment is extrusion.
[11] The process of any one of paragraphs [1] to [3], wherein the mechanical treatment is beating.
[12] The process of any one of paragraphs [1] to [11], further comprising an additional mechanical treatment and second saccharification steps following step (d).
[13] The process of any one of paragraphs [1] to [11], further comprising two additional mechanical treatments and second saccharification steps following step (d).
[14] The process of any one of paragraphs [1] to [13], wherein the lignocellulosic biomass is subjected to one or more pretreatment methods prior to the step (a).
[15] The process of paragraph [14], wherein the one or more pretreatment methods is hot water pretreatment, steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation.
[16] The process of paragraph [14] wherein the one or more pretreatment methods is hot water pretreatment, steam pretreatment, or dilute acid pretreatment.
[17] The process of paragraph [14] wherein the one or more pretreatment methods is hot water pretreatment.
[18] The process of paragraph [14] wherein the one or more pretreatment methods is steam pretreatment.
[19] The process of paragraph [14] wherein the one or more pretreatment methods is dilute acid pretreatment.
[20] The process of any one of paragraphs [1] to [19], further comprising a solids-liquid separation step after the first saccharification of step (b) and before the mechanical treatment of step (c).
[21] The process of paragraph [20], wherein after the mechanical treatment of step (c), the mechanically-treated, partially-hydrolyzed biomass is recombined with the liquor from the solids-liquid separation step.
[22] The process of any one of paragraphs [1] to [21], wherein the cellulase enzyme is a cellobiohydrolase.
[23] The process of any one of paragraphs [1] to [21], wherein the cellulase enzyme is an endoglucanase.
[24] The process of any one of paragraphs [1] to [21], wherein the cellulase enzyme comprises a cellobiohydrolase and an endoglucanase.
[25] The process of any one of paragraphs [1] to [21], wherein the cellulase enzyme comprises an endoglucanase and a beta-glucosidase.
[26] The process of any one of paragraphs [1] to [21], wherein the cellulase enzyme comprises a cellobiohydrolase and a beta-glucosidase.
[27] The process of any one of paragraphs [1] to [21], wherein the cellulase enzyme comprises a cellobiohydrolase, an endoglucanase, and a beta-glucosidase.
[28] The process of any one of paragraphs [22] to [27], wherein the cellulase enzyme further comprises one or more (e.g., several) proteins selected from the group consisting of a polypeptide having cellulolytic enhancing activity, an expansin, a ligninolytic enzyme, an oxidoreductase, a pectinase, a protease, and a swollenin.
[29] The process of any one of paragraphs [22] to [27], wherein the cellulase enzyme further comprises a polypeptide having cellulolytic enhancing activity.
[30] The process of paragraph [29], wherein the polypeptide having cellulolytic enhancing activity is an AA9 (formerly GH61) polypeptide.
[31] The process of any one of paragraphs [1] to [30], wherein the hemicellulase enzyme is an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase or any combination thereof.
[32] The process of any one of paragraphs [1] to [30], wherein the hemicellulase enzyme is a xylanase.
[33] The process of any one of paragraphs [1] to [30], wherein the hemicellulase enzyme is a xylosidase.
[34] The process of any one of paragraphs [1] to [30], wherein the hemicellulase enzyme is a xylanase and a xylosidase.
[35] The process of paragraph [2] wherein step (e) is conducted simultaneously with step (b) and/or step (d) in a simultaneous saccharification and fermentation.
[36] The process of process of paragraph [2], wherein the fermentation product is an alcohol, an alkane, a cycloalkane, an alkene, an amino acid, a gas, isoprene, a ketone, an organic acid, or polyketide.
[37] The process of process of paragraph [2], wherein the fermentation product is ethanol, n-butanol, or isobutanol.
[38] The process of any one paragraphs [1] to [35], wherein the biomass is agricultural residue (sugar cane bagasse, corn stover, wheat straw, barley straw, rice straw, oat straw, canola straw, and soybean stover), herbaceous material (including energy crops), municipal solid waste, pulp and paper mill residue, waste paper, wood (including forestry residue), or any combination thereof.
[39] A process for producing a fermentation product, comprising
   a. pretreatment of lignocellulosic biomass containing cellulose and/or hemicellulose by hot water pretreatment, steam pretreatment, dilute acid pretreatment, wet oxidation, wet explosion pretreatment with organic solvents, biological pretreatment, supercritical CO₂ pretreatment, or ozone pretreatment;
   b. preparation of a biomass-enzyme mixture comprising (i) the pretreated lignocellulosic biomass of step (a) and (ii) an enzyme composition comprising cellulase enzymes and/or hemicellulase enzymes;
   c. a first saccharification of the biomass-enzyme mixture from step (b) for a sufficient time to achieve hydrolysis of at least about 10% of the cellulose and/or hemicellulose and produce partially-hydrolyzed biomass and hydrolysate liquor;
   d. mechanical treatment of the partially-hydrolyzed biomass produced by step (c) to produce mechanically-disrupted, partially-hydrolyzed biomass;
   e. a second saccharification of the mechanically-disrupted, partially-hydrolyzed biomass produced by step (d) for a sufficient time to achieve hydrolysis of at least about 60% of the cellulose and/or hemicellulose to fermentable sugars;
   f. fermenting the fermentable sugars produced in step (e) with one or more fermenting microorganisms to produce the fermentation product; and
   g. recovering the fermentation product from the fermentation.
[40] The process of paragraph [39], wherein the second saccharification of step (e) is conducted without an additional dose of cellulase and/or hemicellulase enzymes.
[41] The process of paragraph [39], wherein the second saccharification of step (e) is conducted with an additional dose of cellulase and/or hemicellulose enzymes.
[42] The process of any one of paragraphs [39] to [41], wherein the mechanical treatment of step (d) is selected from the group consisting of refining, milling, crushing, grinding, shredding, extrusion, beating, or combinations thereof.
[43] The process of any one of paragraphs [39] to [41], wherein the mechanical treatment is refining.
[44] The process of paragraph [43], wherein the refining is conducted so as to provide a refining energy of 50 to 500 kWh per dry tonne of biomass, 50 to 450 kWh per dry tonne of biomass, to 400 kWh per dry tonne of biomass, 50 to 350 kWh per dry tonne of biomass, 50 to 300 kWh per dry tonne of biomass, 100 to 400 kWh per dry tonne of biomass, 100 to 350 kWh per dry tonne of biomass, or 100 to 300 kWh per dry tonne of biomass.
[45] The process of paragraph [39], further comprising one or more additional mechanical treatments and second saccharification steps.
[46] The process of any one of paragraphs [39] to [41], wherein the lignocellulosic biomass is subjected to one or more pretreatment methods prior to the step (a).
[47] The process of paragraph [46], wherein the one or more pretreatment methods is hot water pretreatment, steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation.
[48] The process of paragraph [47] wherein the one or more pretreatment methods is hot water pretreatment, steam pretreatment, or dilute acid pretreatment.
[49] The process of paragraph [47] wherein the one or more pretreatment methods is hot water pretreatment.
[50] The process of paragraph [47] wherein the one or more pretreatment methods is steam pretreatment.
[51] The process of paragraph [47] wherein the one or more pretreatment methods is dilute acid pretreatment.
[52] The process of any one of paragraphs [39] to [51], further comprising a solids-liquid separation step after the first saccharification of step (c) and before the mechanical treatment of step (d).
[53] The process of paragraph [52], wherein after the mechanical treatment of step (d), the mechanically-treated, partially-hydrolyzed biomass is recombined with the liquids from the solids-liquid separation step.
[54] The process of any one of paragraphs [39] to [51], wherein the cellulase enzyme is a cellobiohydrolase.
[55] The process of any one of paragraphs [39] to [51], wherein the cellulase enzyme is an endoglucanase.
[56] The process of any one of paragraphs [39] to [51], wherein the cellulase enzyme comprises a cellobiohydrolase and an endoglucanase.
[57] The process of any one of paragraphs [39] to [51], wherein the cellulase enzyme comprises an endoglucanase and a beta-glucosidase.
[58] The process of any one of paragraphs [39] to [51], wherein the cellulase enzyme comprises a cellobiohydrolase and a beta-glucosidase.
[59] The process of any one of paragraphs [39] to [51], wherein the cellulase enzyme comprises a cellobiohydrolase, an endoglucanase, and a beta-glucosidase.
[60] The process of any one of paragraphs [52] to [59], wherein the cellulase enzyme further comprises one or more (e.g., several) proteins selected from the group consisting of a polypeptide having cellulolytic enhancing activity, an expansin, a ligninolytic enzyme, an oxidoreductase, a pectinase, a protease, and a swollenin.
[61] The process of any one of paragraphs [52] to [59], wherein the cellulase enzyme further comprises a polypeptide having cellulolytic enhancing activity.
[62] The process of paragraph [61], wherein the polypeptide having cellulolytic enhancing activity is an AA9 (formerly GH61) polypeptide.
[63] The process of any one of paragraphs [39] to [62], wherein the hemicellulase enzyme is an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase or any combination thereof.
[64] The process of any one of paragraphs [39] to [62], wherein the hemicellulase enzyme is a xylanase.
[65] The process of any one of paragraphs [39] to [62], wherein the hemicellulase enzyme is a xylosidase.
[66] The process of any one of paragraphs [39] to [62], wherein the hemicellulase enzyme is a xylanase and a xylosidase.
[67] The process of any one of paragraphs [39] to [62], wherein the mechanical treatment is milling.
[68] The process of any one of paragraphs [39] to [62], wherein the mechanical treatment is crushing or grinding.
[69] The process of any one of paragraphs [39] to [62], wherein the mechanical treatment is extrusion.
[70] The process of any one of paragraphs [39] to [62], wherein the mechanical treatment is beating.
[71] The process of any one of paragraph [1] to [70], wherein the the biomass is agricultural residue (sugar cane bagasse, corn stover, wheat straw, barley straw, rice straw, oat straw, canola straw, and soybean stover), herbaceous material (including energy crops), municipal solid waste, pulp and paper mill residue, waste paper, wood (including forestry residue), or any combination thereof.
[72] The process of any one of paragraph [1] to [71], wherein the biomass is sugar cane bagasse, corn stover, or wheat straw.
[73] The process of any one of paragraph [1] to [71], wherein the biomass is herbaceous material (including energy crops).

1. A process for producing fermentable sugars from biomass, comprising:
a. preparation of a biomass-enzyme mixture comprising (i) pretreated lignocellulosic biomass containing cellulose and/or hemicellulose and (ii) an enzyme composition comprising cellulase and/or hemicellulase enzymes;
b. a first saccharification comprising incubation of the biomass-enzyme mixture from step (a) for a sufficient time to achieve hydrolysis of at least about 10% of the cellulose and/or hemicellulose and to produce partially-hydrolyzed biomass and hydrolysate liquor;
c. mechanical treatment of the partially-hydrolyzed biomass produced by step (b) to produce mechanically-disrupted, partially-hydrolyzed biomass; and
d. a second saccharification comprising incubation of the mechanically-disrupted, partially-hydrolyzed biomass produced by step (c) for a sufficient time to achieve hydrolysis of at least about 60% to about 100% of the cellulose and/or hemicellulose present in the pretreated lignocellulosic biomass to fermentable sugars.

2. The process of claim 1, wherein the second saccharification of step (d) is conducted without an additional dose of cellulase and/or hemicellulase enzymes.

3. The process of claim 1, wherein the second saccharification of step (d) is conducted with an additional dose of cellulase and/or hemicellulase enzymes.

4. The process of claim 1, wherein the mechanical treatment is selected from the group consisting of disk refining, milling, crushing, grinding, shredding, extrusion, beating, or combinations thereof.

5. The process of claim 4, wherein the mechanical treatment is refining.

6. The process of claim 5, wherein the refining is conducted so as to provide a refining energy of from about 50 to about 500 kWh per dry tonne of biomass.

7. The process of claim 1, further comprising one or more additional mechanical treatments and second saccharification steps.

8. The process of claim 1, wherein the lignocellulosic biomass is subjected to one or more pretreatment methods prior to the step (a).

9. The process of claim 8, wherein the one or more pretreatment methods is hot water pretreatment, steam pretreatment, dilute acid pretreatment, wet oxidation, wet explosion pretreatment with organic solvents, biological pretreatment, supercritical CO₂ pretreatment, supercritical H₂O pretreatment, ozone pretreatment, ionic liquid pretreatment, or ultrasound, microwave, or gamma irradiation.

10. The process of claim 1, further comprising a solids-liquid separation step after the first saccharification of step (b) and before the mechanical treatment of step (c).

11. The process of claim 10, wherein after the mechanical treatment of step (c), the mechanically-treated, partially-hydrolyzed biomass is recombined with the liquids from the solids-liquid separation step.

12. The process of claim 1, wherein the cellulase enzyme is a cellobiohydrolase, an endoglucanase, a beta-glucosidase, or mixtures thereof.

13. The process of claim 1, wherein the cellulase enzyme composition further comprises one or more (e.g., several) proteins selected from the group consisting of a polypeptide having cellulolytic enhancing activity, an expansin, a ligninolytic enzyme, an oxidoreductase, a pectinase, a protease, and a swollenin.

14. The process of claim 13, wherein the cellulase enzyme composition further comprises a polypeptide having cellulolytic enhancing activity.

15. The process of claim 14, wherein the polypeptide having cellulolytic enhancing activity is an Auxilliary Activity 9 (AA9) polypeptide.

16. The process of claim 1, wherein the hemicellulase enzyme is an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, a xylosidase, or any combination thereof.

17. A process for producing a fermentation product, comprising:
a. preparation of a biomass-enzyme mixture comprising (i) pretreated lignocellulosic biomass containing cellulose and/or hemicellulose and (ii) an enzyme composition comprising cellulase enzymes and/or hemicellulase enzymes;
b. a first saccharification of the biomass-enzyme mixture from step (a) for a sufficient time to achieve hydrolysis of at least about 10% of the cellulose and/or hemicellulose and to produce partially-hydrolyzed biomass and hydrolysate liquor;
c. mechanical treatment of the partially-hydrolyzed biomass produced by step (b) to produce mechanically-disrupted, partially-hydrolyzed biomass;
d. a second saccharification of the mechanically-disrupted, partially-hydrolyzed biomass produced by step (c) for a sufficient time to achieve hydrolysis of at least about 60% of the cellulose and/or hemicellulose present in the pretreated lignocellulosic biomass to fermentable sugars;
e. fermenting the fermentable sugars produced in step (d) with one or more fermenting microorganisms to produce a fermentation product; and
f. recovering the fermentation product from the fermentation.

18. The process of claim 17, wherein step (e) is conducted simultaneously with step (b) and/or step (d) in a simultaneous saccharification and fermentation.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A process for producing a fermentation product, comprising:
a. pretreatment of lignocellulosic biomass containing cellulose and/or hemicellulose by hot water pretreatment, steam pretreatment, dilute acid pretreatment, wet oxidation, wet explosion pretreatment with organic solvents, biological pretreatment, supercritical CO₂ pretreatment, or ozone pretreatment to form a pretreated lignocellulosic biomass;
b. preparation of a biomass-enzyme mixture comprising (i) the pretreated lignocellulosic biomass of step (a) and (ii) an enzyme composition comprising cellulase enzymes and/or hemicellulase enzymes;
c. a first saccharification of the biomass-enzyme mixture from step (b) for a sufficient time to achieve hydrolysis of at least about 10% of the cellulose and/or hemicellulose and to produce partially-hydrolyzed biomass and hydrolysate liquor;
d. mechanical treatment of the partially-hydrolyzed biomass produced by step (c) to produce mechanically-disrupted, partially-hydrolyzed biomass;
e. a second saccharification of the mechanically-disrupted, partially-hydrolyzed biomass produced by step (d) for a sufficient time to achieve hydrolysis of at least about 60% of the cellulose and/or hemicellulose to fermentable sugars;
f. fermenting the fermentable sugars produced in step (e) with one or more fermenting microorganisms to produce a fermentation product; and
g. recovering the fermentation product from the fermentation.

31. The process of claim 1, wherein the biomass is agricultural residue (sugar cane bagasse, corn stover, wheat straw, barley straw, rice straw, oat straw, canola straw, and soybean stover), herbaceous material (including energy crops), municipal solid waste, pulp and paper mill residue, waste paper, wood (including forestry residue), or any combination thereof.

32. (canceled)

33. (canceled)
